(19) United States
(12) Patent Application Publication
(10) Pub. No.: US 2026/0083826 A1
(43) Pub. Date: Mar. 26, 2026

(54) NEW SYNTHETIC DRUGS FOR TREATING ALZHEIMER'S DISEASE

(71) Applicant: OKINAWA INSTITUTE OF SCIENCE AND TECHNOLOGY SCHOOL CORPORATION, Okinawa (JP)

(72) Inventors: Tetsuya HORI, Okinawa (JP); Tomoyuki TAKAHASHI, Okinawa (JP); Zacharie TAOUFIQ, Okinawa (JP); Kohji TAKEI, Okayama (JP); Hiroshi YAMADA, Okayama (JP)

(73) Assignee: OKINAWA INSTITUTE OF SCIENCE AND TECHNOLOGY SCHOOL CORPORATION, Okinawa (JP)

(21) Appl. No.: 19/111,601

(22) PCT Filed: Sep. 13, 2023
(86) PCT No.: PCT/JP2023/033289

§ 371 Date: Mar. 13, 2025

(30) Foreign Application Priority Data
Sep. 13, 2022 (JP) 2022-145546

(51) Int. Cl. A61K 38/46; A61P 25/28; G01N 33/573

(52) U.S. Cl. CPC A61K 38/46; A61P 25/28; C12Y 306/05005; G01N 33/573; G01N 2333/914; G01N 2500/02

(57) ABSTRACT

The present invention aims to provide a novel agent for treating Alzheimer's disease, a method for treating Alzheimer's disease, a method for screening for a candidate substance for a therapeutic drug for Alzheimer's disease, and the like. The present invention is a prophylactic and/or therapeutic agent for Alzheimer's disease comprising a peptide corresponding to dynamin 1. The peptide preferably corresponds to dynamin 1-pleckstrin-homology domain or dynamin 1-proline rich domain. In addition, the peptide is preferably encapsulated in nano-particles or linked to a peptide sequence that improve delivery of the peptide into the brain.

### TECHNICAL FIELD

The present invention relates to a synthetic drug for treating Alzheimer's disease, a method for treating Alzheimer's disease, and a method for screening for a candidate substance for a therapeutic drug for Alzheimer's disease.

### BACKGROUND ART

Alzheimer's disease (AD) is a progressive dementia that starts with decrease of short term memory and mild learning disability, develops higher brain dysfunction, particularly visuospatial agnosia, ideational apraxia, constructive apraxia and the like, and finally reaches movement disorder and so-called personality destruction, for which a method that can completely cure Alzheimer's disease has not been found to date. There are predicted to be 2.4 million patients with Alzheimer's disease in the world in 2040, and the importance of a novel treatment method therefore is increasing.

The microtubule (MT) binding protein tau assembles and stabilizes MTs (1, 2) mainly in axonal compartments (3, 4). Phosphorylation of tau proteins reduces their binding affinity (5, 6), thereby shifting the equilibrium from MT-bound form to soluble free form (7). Soluble tau proteins also exist in dynamic equilibrium between phosphorylated and dephosphorylated forms (8) as well as between soluble and aggregated forms. When the cytosolic tau concentration is elevated, monomeric tau undergoes oligomerization and eventually precipitates into neurofibrillary tangles (NFT) (9-11), which is a hallmark of tauopathies, including Alzheimer's disease (AD), frontotemporal dementia with Parkinsonism-17 (FTDP-17) and progressive supranuclear palsy (PSP) (2, 7, 8). Although the NFT density can correlate with the degree of AD progression (2, 7, 12), soluble tau protein levels are more closely linked to disease progression and cognitive decline (13, 14).

Genetic ablation of tau shows little abnormal phenotype (15-17), presumably due to compensation by other MT-associated proteins (15). Instead, tau ablation can prevent amyloid β-induced impairments of mitochondrial transport (16) or memory defects (18, 19). Thus, loss of tau function due to its dissociation from MTs is unlikely to be an important cause of neuronal dysfunction in AD (8, 12).

In postmortem brains of both AD patients and intact humans, tau is present in synaptosomes (20, 21). In a transgenic mice AD model, soluble tau is accumulated in the hippocampal nerve terminal zone (22, 23). Both in vivo and in culture models of tauopathy, tau is released from axon terminals upon KCl stimulation in a Ca²⁺ -dependent manner, like neurotransmitters (24, 25). Tau oligomers produced by released tau triggers endogenous tau seeding in neighboring neurons, thereby causing trans-synaptic propagations (22, 26).

FTDP tauopathy model mice that overexpressed with mutant tau are widely used to examine tau toxicities on synaptic plasticity (27-29), memory formation (28, 30) as well as on synaptic vesicle transport (31, 32). In contrast to FTDP, which is a rare familial disease associated with tau mutation, AD is a widespread sporadic disease unassociated with tau mutation, but the expression level of WT tau being crucial. As AD models, the effects of WT tau overexpression have been examined in culture cells (33-36) or in Drosophila (37), where impaired axonal transports associated with increased MT density were found. These observations suggest that WT tau can be detrimental when its levels are elevated (35, 36). However, unlike FTDP tau mutant, it is unknown whether elevated soluble WT tau can affect mammalian central synaptic transmission.

### CITATION LIST

### Non Patent Literature

NPL 1: K. R. Brunden, J. Q. Trojanowski, V. M. Lee, Advances in tau-focused drug discovery for Alzheimer's disease and related tauopathies. Nat Rev Drug Discov 8, 783-793 (2009).
NPL 2: V. M. Lec, K. R. Brunden, M. Hutton, J. Q. Trojanowski, Developing therapeutic approaches to tau, selected kinases, and related neuronal protein targets. Cold Spring Harb Perspect Med 1, a006437 (2011).
NPL 3: L. I. Binder, A. Frankfurter, L. I. Rebhun, The distribution of tau in the mammalian central nervous system. J Cell Biol 101, 1371-1378 (1985).
NPL 4: A. Kubo et al., Distribution of endogenous normal tau in the mouse brain. J Comp Neurol 527, 985-998 (2019).
NPL 5: J. Biernat, N. Gustke, G. Drewes, E. M. Mandelkow, E. Mandelkow, Phosphorylation of Ser262 strongly reduces binding of tau to microtubules: distinction between PHF-like immunorcactivity and microtubule binding. Neuron 11, 153-163 (1993). NPL 6: D. N. Drechsel, A. A. Hyman, M. H. Cobb, M. W. Kirschner, Modulation of the dynamic instability of tubulin assembly by the microtubule-associated protein tau. Mol Biol Cell 3, 1141-1154 (1992).
NPL 7: C. Ballatore, V. M. Lec, J. Q. Trojanowski, Tau-mediated neurodegeneration in Alzheimer's disease and related disorders. Nat Rev Neurosci 8, 663-672 (2007). NPL 8: M. G. Spillantini, M. Goedert, Tau pathology and neurodegeneration. Lancet Neurol 12, 609-622 (2013).
NPL 9: S. G. Greenberg, P. Davies, A preparation of Alzheimer paired helical filaments that displays distinct tau proteins by polyacrylamide gel electrophoresis. Proc Natl Acad Sci USA 87, 5827-5831 (1990).
NPL 10: I. Grundke-Iqbal et al., Abnormal phosphorylation of the microtubule-associated protein tau (tau) in Alzheimer cytoskeletal pathology. Proc Natl Acad Sci USA 83, 4913-4917 (1986).
NPL 11: H. Yoshida, Y. Ihara, Tau in paired helical filaments is functionally distinct from fetal tau: assembly incompetence of paired helical filament-tau. J Neurochem 61, 1183-1186 (1993).
NPL 12: M. Morris, S. Maeda, K. Vossel, L. Mucke, The many faces of tau. Neuron 70, 410-426 (2011).
NPL 13: J. Gotz, L. M. Ittner, M. Fandrich, N. Schonrock, Is tau aggregation toxic or protective: a sensible question in the absence of sensitive methods? J Alzheimers Dis 14, 423-429 (2008).
NPL 14: D. J. Koss et al., Soluble pre-fibrillar tau and beta-amyloid species emerge in early human Alzheimer's disease and track disease progression and cognitive decline. Acta Neuropathol 132, 875-895 (2016).
NPL 15: A. Harada et al., Altered microtubule organization in small-calibre axons of mice lacking tau protein. Nature 369, 488-491 (1994).
NPL 16: K. A. Vossel et al., Tau reduction prevents Abeta-induced defects in axonal transport. Science 330, 198 (2010).
NPL 17: A. Yuan, A. Kumar, C. Peterhoff, K. Duff, R. A. Nixon, Axonal transport rates in vivo are unaffected by tau deletion or overexpression in mice. J Neurosci 28, 1682-1687 (2008).
NPL 18: L. M. Ittner et al., Dendritic function of tau mediates amyloid-beta toxicity in Alzheimer's disease mouse models. Cell 142, 387-397 (2010).
NPL 19: E. D. Roberson et al., Reducing endogenous tau ameliorates amyloid beta-induced deficits in an Alzheimer's disease mouse model. Science 316, 750-754 (2007). NPL 20: J. A. Fein et al., Co-localization of amyloid beta and tau pathology in Alzheimer's disease synaptosomes. Am J Pathol 172, 1683-1692 (2008).
NPL 21: H. C. Tai et al., The synaptic accumulation of hyperphosphorylated tau oligomers in Alzheimer disease is associated with dysfunction of the ubiquitin-proteasome system. Am J Pathol 181, 1426-1435 (2012).
NPL 22: A. de Calignon et al., Propagation of tau pathology in a model of early Alzheimer's disease. Neuron 73, 685-697 (2012).
NPL 23: L. Liu et al., Trans-synaptic spread of tau pathology in vivo. PLOS One 7, c31302 (2012).
NPL 24: A. M. Pooler, E. C. Phillips, D. H. Lau, W. Noble, D. P. Hanger, Physiological release of endogenous tau is stimulated by neuronal activity. EMBO Rep 14, 389-394 (2013).
NPL 25: K. Yamada et al., Neuronal activity regulates extracellular tau in vivo. J Exp Med 211, 387-393 (2014).
NPL 26: J. L. Guo, V. M. Lee, Seeding of normal Tau by pathological Tau conformers drives pathogenesis of Alzheimer-like tangles. J Biol Chem 286, 15317-15331 (2011). NPL 27: M. Polydoro et al., Soluble pathological tau in the entorhinal cortex leads to presynaptic deficits in an early Alzheimer's disease model. Acta Neuropathol 127, 257-270 (2014).
NPL 28: A. Sydow et al., Tau-induced defects in synaptic plasticity, learning, and memory are reversible in transgenic mice after switching off the toxic Tau mutant. J Neurosci 31, 2511-2525 (2011).
NPL 29: Y. Yoshiyama et al., Synapse loss and microglial activation precede tangles in a P301S tauopathy mouse model. Neuron 53, 337-351 (2007).
NPL 30: K. Santacruz et al., Tau suppression in a neurodegenerative mouse model improves memory function. Science 309, 476-481 (2005).
NPL 31: J. McInnes et al., Synaptogyrin-3 Mediates Presynaptic Dysfunction Induced by Tau. Neuron 97, 823-835 e828 (2018).
NPL 32: L. Zhou et al., Tau association with synaptic vesicles causes presynaptic dysfunction. Nat Commun 8, 15295 (2017).
NPL 33: A. Ebneth et al., Overexpression of tau protein inhibits kinesin-dependent trafficking of vesicles, mitochondria, and endoplasmic reticulum: implications for Alzheimer's disease. J Cell Biol 143, 777-794 (1998)
NPL 34: K. Shahpasand et al., Regulation of mitochondrial transport and inter-microtubule spacing by tau phosphorylation at the sites hyperphosphorylated in Alzheimer's disease. J Neurosci 32, 2430-2441 (2012).
NPL 35: K. Stamer, R. Vogel, E. Thies, E. Mandelkow, E. M. Mandelkow, Tau blocks traffic of organelles, neurofilaments, and APP vesicles in neurons and enhances oxidative stress. J Cell Biol 156, 1051-1063 (2002).
NPL 36: E. Thies, E. M. Mandelkow, Missorting of tau in neurons causes degeneration of synapses that can be rescued by the kinase MARK2/Par-1. J Neurosci 27, 2896-2907 (2007).
NPL 37: A. Mudher et al., GSK-3beta inhibition reverses axonal transport defects and behavioural phenotypes in Drosophila. Mol Psychiatry 9, 522-530 (2004).

### SUMMARY OF INVENTION

### Technical Problem

The present invention aims to provide a novel agent for treating Alzheimer's disease, a method for treating Alzheimer's disease, a method for screening for a candidate substance for a therapeutic drug for Alzheimer's disease, and the like.

### Solution to Problem

We addressed the question whether elevated soluble WT tau can affect mammalian central synaptic transmission or not using the giant nerve terminal calyx of Held visualized in slices from mice brainstem, where axonal MTs extended into the depth of terminals (38). In this presynaptic terminal, we loaded recombinant WT h-tau from a whole-cell patch pipette at fixed concentrations to model the elevation of WT tau associated with AD and found that WT h-tau newly assembled MTs and strongly impaired synaptic transmission. Capacitance measurements indicated that the primary target of WT h-tau is vesicle endocytosis. Immunocytochemical image analysis after cell permeabilization revealed an increase in the MT-bound fraction of the endocytic GTPase dynamin in WT h-tau-loaded terminals. Since the endocytic key protein dynamin is a MT-binding protein (39), dynamin is likely sequestered by newly assembled MTs. Out of screening, we found that a synthetic dodecapeptide corresponding to amino acids 560-571 of dynamin 1 inhibited MT-dynamin interaction. When we co-loaded this peptide “PHDP5” with WT h-tau, its toxicities on vesicle endocytosis as well as on synaptic transmission were rescued. Thus, we propose a novel synaptic dysfunction mechanism underlying AD, in which WT tau-induced over-assembly of MTs depletes dynamins, thereby impairing vesicle endocytosis and synaptic transmission. The inventions completed by the above new findings are as follows.

The present invention includes the following embodiments:

(1)

A prophylactic and/or therapeutic agent for Alzheimer's disease comprising a peptide corresponding to dynamin 1.

(2)

The prophylactic and/or therapeutic agent for Alzheimer's disease according to (1), wherein the peptide corresponds to dynamin 1-pleckstrin-homology domain or dynamin 1-proline rich domain.

(3)

The prophylactic and/or therapeutic agent for Alzheimer's disease according to (1), wherein the peptide comprises an amino acid sequence selected from the group consisting of Sequence ID Numbers 7 to 9, an amino acid sequence having one or more conservative amino acid substitutions in the amino acid sequence selected from the group consisting of Sequence ID Numbers 7 to 9 or an amino acid sequence having at least 80% amino acid sequence identity with the amino acid sequence selected from the group consisting of Sequence ID Numbers 7 to 9.

(4)

The prophylactic and/or therapeutic agent for Alzheimer's disease according to (1), wherein the peptide is encapsulated in nano-particles for improved delivery of the peptide into the brain.

(5)

The prophylactic and/or therapeutic agent for Alzheimer's disease according to (1), wherein the peptide is linked to a peptide sequence that improves delivery of the peptide into the brain.

(6)

The prophylactic and/or therapeutic agent for Alzheimer's disease according to (1), wherein the peptide sequence that improves delivery of the peptide into the brain is selected from the group consisting of Sequence ID Numbers 10 to 15.

(7)

The prophylactic and/or therapeutic agent for Alzheimer's disease according to (1), wherein the peptide is fused to or conjugated with a compound that improves delivery of the peptide into the brain.

(8)

A prophylactic and/or therapeutic agent for Alzheimer's disease comprising an inhibitor of microtubule-dynamin 1 binding.

(9)

The prophylactic and/or therapeutic agent for Alzheimer's disease according to (8), wherein the inhibitor is an isolated monoclonal antibody that binds to dynamin 1.

(10)

The prophylactic and/or therapeutic agent for Alzheimer's disease according to (8), wherein the inhibitor is an isolated monoclonal antibody that binds to dynamin 1-pleckstrin-homology domain or dynamin 1-proline rich domain.

(11)

An isolated monoclonal antibody that inhibits microtubule-dynamin 1 binding, wherein the monoclonal antibody is against dynamin 1.

(12)

The isolated monoclonal antibody that inhibits microtubule-dynamin 1 binding according to (11), wherein the monoclonal antibody is against dynamin 1-pleckstrin-homology domain or dynamin 1-proline rich domain.

(13)

An isolated monoclonal antibody that inhibits microtubule-dynamin 1 binding according to (11), wherein the monoclonal antibody is against the peptide which comprises an amino acid sequence selected from the group consisting of Sequence ID Numbers 7 to 9, an amino acid sequence having one or more conservative amino acid substitutions in the amino acid sequence selected from the group consisting of Sequence ID Numbers 7 to 9 or an amino acid sequence having at least 80% amino acid sequence identity with the amino acid sequence selected from the group consisting of Sequence ID Numbers 7 to 9.

(14)

A method for screening for substances effective for the treatment of Alzheimer's disease, comprising a step of measuring an activity of the test substances to inhibit the binding of microtubules and dynamin 1.

### Advantageous Effects of Invention

The present invention provides a novel agent for treating Alzheimer's disease, a method for treating Alzheimer's disease, a method for screening for a candidate substance for a therapeutic drug for Alzheimer's disease, and the like.

### BRIEF DESCRIPTION OF DRAWINGS

FIG. 1 WT h-tau loaded in presynaptic terminals inhibited excitatory synaptic transmission (A) In simultaneous pre- and postsynaptic whole-cell recordings, intra-terminal infusion of WT h-tau at 10 μM (blue filled circles) or 20 μM (black filled circles), from a tube in a presynaptic patch pipette (top illustration), caused a con-centration-dependent rundown of EPSCs evoked by presynaptic action potentials at 1 Hz. In the time plots, EPSC amplitudes averaged from 60 events are sampled for data points and normalized to the mean amplitude of baseline EPSCs before h-tau infusion. Sample records of EPSCs 5 min before (i) and 30 min after (ii) tau infusion are superimposed and shown on the left panels. The EPSC amplitude remaining 30 min after infusion was 23±9% and 65±5%, respectively for 10 μM and 20 μM h-tau (means and SEMs, 6 synapses from 6 slices, p<0.01 in paired t-test between before and after h-tau infusion). Infusion of MT-binding site-deleted h-tau mutant (del-MTBD, 20 μM, FIG. 10A) had no effect on the EPSC amplitude (open circles, sample EPSC traces shown on the left bottom panel). (B) The amplitude of EPSCs evoked at 0.1 Hz remained unchanged after h-tau infusion (85%+12%, 5 synapses from 5 slices, p=0.22 in paired t-test). Sample records of EPSCs before (i) and 30 min after (ii) h-tau infusion at 0.1 Hz are superimposed on the left panel. (C) Taxol (1 μM) caused activity-dependent rundown of EPSC amplitude to 41.4±12% at 1 Hz (p<0.01, 5 synapses from 5 slices), but remained unchanged when stimulated at 0.1 Hz (105±3.0%, open circles, 4 synapses from 4 slices). Sample records of EPSCS at 0.1 Hz and 1 Hz are superimposed on the left panels.

FIG. 2 Inhibition of SV endocytosis is the primary effect of WT h-tau loading (A) Exo-endocytic membrane capacitance changes in presynaptic terminals without (Control) or after direct loading of WT h-tau (20 μM; Tau). WT h-tau was directly loaded by diffusion into a terminal from a whole-cell patch pipette (illustration). Capacitance traces were sampled from (i) 10, (ii) 20 and (iii) 30 min after patch membrane rupture (superimposed). Left panel, non-loading control. Right panel, WT h-tau-loaded terminal. Capacitance changes were evoked every 2 min by Ca²⁺ currents induced by a 20-ms depolarizing pulse (not shown). (B) Time plots of endocytic rate (left panel), exocytic magnitude (middle panel) and presynaptic Ca²⁺ current charge (right panel). Data points represent averaged values from 5 events from 4 min before and 4 min after the time points. In calyceal terminals, 20 min after patch membrane rupture with a pipette containing WT h-tau (filled circles; Tau), endocytic rate was significantly prolonged (p<0.05 compared to controls, open circles, repeated-measures two-way ANOVA with post-hoc Scheffe test, n=5 from 5 slices), whereas exocytic magnitude remained similar to controls (p=0.45). 30 min after rupture, endocytic rate was further prolonged (p<0.01) and exocytic magnitude became significantly less than controls (p<0.05). Ca²⁺ current charge (Q_{Ca}) remained unchanged throughout recording.

FIG. 3 The MT assembly blocker nocodazole prevented tau-induced block of SV endocytosis and EPSC rundown (A) Concentration-dependent inhibitory effects of nocodazole on MT assembly in tubulin polymerization assay. MT assembly by ON4R h-tau (20 μM) in the absence (pink symbols and a fitting line) or presence of nocodazole at 1 μM (blue), 10 μM (red), 20 μM (purple) and 50 μM (orange). Data points and error bars in all graphs represent means and SEMs (n=3). (B) Nocodazole prevented h-tau-induced block of SV endocytosis. Presynaptic membrane capacitance changes (superimposed traces) 25 min after loading h-tau alone (20 μM, red trace), h-tau and nocodazole (20 μM, blue), nocodazole alone (20 μM, green) and controls with no loading (black). Bar graphs indicate endocytic rates in non-loading controls (Ctr, black, 6 terminals from 6 slices), h-tau loaded terminals (Tau, red, 8 terminals from 8 slices), co-loading of nocodazole with h-tau (N+T, blue, 7 terminals from 7 slices) and nocodazole alone (Noc, green, 8 terminals from 8 slices). Nocodazole co-loading fully prevented endocytic block by h-tau (p<0.01, between Tau and N-T) to control level (one-way ANOVA with Scheffe post hoc test). (C) Nocodazole prevented EPSC rundown caused by WT h-tau. Nocodazole (20 μM) co-loaded with WT h-tau (20 μM) prevented EPSC rundown (filled circles, 4 synapses from 4 slices, p<0.01, unpaired t-test). Data of WT h-tau effect on EPSCs (FIG. 1A) is shown as a red dashed line for comparison. Nocodazole alone (20 μM) had no effect on EPSC amplitude throughout (open circles, 4 synapses from 4 slices).

FIG. 4 WT h-tau assembled MTs and increased bound-form dynamins in calyccal terminals (A) Immunofluorescence images of brainstem slices showing loaded WT h-tau (green, left, arrowhead) labeled with anti h-tau/AlexaFluor-488 antibodies (green, left panel), newly assembled MTs labeled with anti β3-tubulin/AlexaFluor-647 antibodies (magenda, middle) and increased bound-form dynamin labeled with anti dynamin1/AlexaFluor-568 antibodies (red, right panel). (B) Bar graphs showing immunofluorescence intensities of h-tau (green), β3-tubulin (magenda) and dynamin (red) relative to controls with no loading (black bars). WT h-tau loading significantly increased β3-tubulin (p=0.0105) and dynamin1 (p=0.0109) intensity in terminals compared to control terminals without WT h-tau loading (n=5 terminals from 5 slices for each data set, two-tailed unpaired t-test with Welch's correction; * p<0.05, *** p<0.001).

FIG. 5 dynamin 1 PH domain peptide inhibited MT-dynamin 1 binding and prevented endocytic slowing and EPSC rundown caused by WT h-tau (A) Top, Partial amino acid sequence of PH domain of mouse dynamin 1 indicating the sequence of the synthetic dodecapeptide PHDP5 (560-571). Left, SDS-PAGE of MT-dynamin 1 binding assay. S, supernatant; P, precipitates. Dyn1, dynamin 1. Right, quantification of MT-dynamin 1 interaction. The bars indicate the percentage of dynamin 1 found in precipitates relative to total amount. PHDP5 significantly inhibited MT-dynamin 1 interaction (**<0.01, n=3). (B) Presynaptic membrane capacitance records (superimposed) after loading h-tau alone (20 μM, red trace, taken from FIG. 4B), h-tau co-loaded with DPHP5 (0.25 mM, blue) or scrambled DPHP5 (SDPHP5, green). DPHP5 alone (0.25 mM, black trace, 7 terminals from 7 slices) had no effect on capacitance changes compared to non-loading terminal controls (taken from FIG. 3B). Bar graphs of endocytic rates (middle panel) indicate significant difference (p<0.05, 7 terminals from 7 slices) between tau (red bar, 8 terminals from 8 slices) and DPHP5+tau (blue, 7 terminals from 7 slices) as well as between SDPHP5+tau (blue) and DPHP5+tau (green, n=6 terminals from 6 slices). The magnitudes of exocytic capacitance changes were not significantly different between the groups, recorded 25 min after rupture). (C) DPHP5 attenuated h-tau induced EPSC rundown. The EPSC rundown after h-tau infusion (20 μM, red dashed line; data taken from FIG. 1A) was attenuated by DPHP5 (1 mM) co-loaded with h-tau (filled circles) but not by scrambled DPHP5 peptide (SDPHP5, open triangles, 1 mM). DPHP5 alone (1 mM, open circles) had no effect on EPSC amplitude throughout. Bar graphs indicate EPSC amplitude (normalized to that before infusion) 30 min after infusion. Significant difference (p<0.01) between tau and tau+DPHP5, between tau+DPHP5 and tau+SDPHP5. The difference between DPHP5 alone and DPHP5+tau was not significant (p=0.09), indicating the partial antagonistic effect of DPHP5 against h-tau-induced EPSC rundown.

FIG. 6A Firstly, MTs were prepared from tubulin and stabilized by taxol. And then taxol stabilized MTs and Dyn1 were incubated with/without PHDP5. Using ultracentrifugation to separate supernatant (unbinding protein) and pellet (bound protein) fractions. Samples were analyzed by SDS-PAGE gel and visualized by SYPRO Orange staining. Results showed % of Dyn1 in pellet fraction has no difference between supernatant and pellet fraction after incubation with PHDP5. This may cause by competitive binding of Dyn1/PHDP5 to MTs.

FIG. 6B We changed the incubation order that MTs were mixed with PHDP5 and then incubated with Dyn1. Results showed % of Dyn1 was decreased in pellet fraction under PHDP5 existing.

FIG. 7A The results showed % of Dyn1 in pellet fraction was lower when MTs incubated with 3 mM of PHDP5 than 1 mM. Lower % of Dyn1 in pellet fraction reveals the potential ability to block MT-dynamin interaction.

FIG. 7B When MTs incubated with 1 mM of PHDP5, shorter incubation period with Dyn1 (10 min) showed lower % of Dyn1 in pellet fraction. Without PHDP5, there is no difference between each time point.

FIG. 7C When MTs incubated with 3 mM of PHDP5, incubation with Dyn1 for 5 min showed lower % of Dyn1 in pellet fraction.

FIG. 7D Under the same condition, several FITC-labeled PHDP5 candidates and its' scrambled control were also tested. No. 1 candidate showed lower % of Dyn1 than PHDP5. However, more tests in our unit are needed.

FIG. 8A Statistic results of figure S4

FIG. 8B Statistic results of figure S4

FIG. 9 Electron images of negatively stained microtubules with dynamin1 in the presence or absence of PHDP5. MT-dynamin1+FP5 #3 seems to stabilize MT-dynamin1 interaction.

FIG. 10 Tubulin polymerization assay for purified ON4R WT h-tau and MT-binding region-deleted mutant (A) Left panel, schematic drawings of WT ON4R h-tau (a) and h-tau deletion mutant lacking the MT-binding region (del-MTBD, b). Right panel, purified recombinant WT ON4R h-tau (a) and del-MTBD (b) in SDS-PAGE with molecular markers (M) on the left lane (B) In vitro tubulin polymerization assay, showing MT assembly by WT h-tau (10 μM, open circles) or taxol (1 μM, filled triangles), but not by del-MTBD (10 μM, open squares) or tubulin alone (filled circles). Data points and bars represent means and SEMs (n=3).

FIG. 11 PHDP5 strongly inhibited dynamin 1 binding to microtubules (A) Peptide sequences in mouse dynamin 1 used in the DN peptide screening. In total, 22 peptides covering the PH domain and the proline-rich domain were synthesized. (B) SDS-PAGE of the microtubule binding assay, showing that PHDP5 strongly inhibits the MT-dynamin 1 binding (“+Pep5” in the top right panel). Since 5 peptides (peptide6, peptide8, peptide9, peptide 10, peptide11) were insoluble in water or dimethyl sulfoxide, their effects were not tested. (C) Effects of the synthetic peptides at 1 mM on MT-dynamin1 binding. Bar graphs indicate the percentage of dynamin 1 found in precipitates relative to total amount.

### DESCRIPTION OF EMBODIMENTS

Unless otherwise noted, all terms in the present invention have the same meaning as commonly understood by one with ordinary skill in the art to which this disclosure belongs. The singular terms “a,” “an,” and “the” include plural referents unless context indicates otherwise. Similarly, the word “or” is intended to include “and” unless the context indicates otherwise. In this specification, molecular biological techniques can be performed by methods described in general experimental manuals known to those skilled in the art or by methods similar thereto, unless otherwise specified.

In the present invention, “corresponding to” or “corresponds to” means that what precedes it corresponds to a part or the whole of the molecule, domain, etc. For example, “peptide corresponding to dynamin 1” means that the peptide corresponds to a part or the whole of dynamin 1. It may or may not be completely identical, and it may be the peptide having at least 80%, preferably at least 85%, more preferably at least 90%, and most preferably at least 95% sequence identity to dynamin 1.

The inventors have shown that tau, a protein involved in Alzheimer's disease that binds to microtubules, reduces synaptic signaling in the mouse brain. Tau proteins produced in nerve cells bind to microtubules and are involved in their polymerization. Normally, the tau protein is either attached to microtubules or dissolved in the fluid inside the cell. However, in some neurological disorders, particularly Alzheimer's disease, the concentration of soluble tau protein becomes excessive in certain areas of the brain and condenses to form insoluble structures called neurofibrillary tangles. The inventors investigated the effects of high concentrations of soluble tau protein on signal transmission at the calyx (calyx) synapses of Held, the largest synapses in the mammalian brain. Injection of soluble tau protein into the presynaptic end of the Held calyx synapse in mice resulted in a marked reduction in the generation of electrical signals in postsynaptic cells. Fluorescent labeling of tau protein and microtubules confirmed that injecting tau protein caused many microtubules to polymerize anew in the presynaptic terminals. However, when a mutant tau protein lacking the binding site involved in microtubule polymerization was injected instead, synaptic signaling was not affected.

It was found that the increase in tau protein decreased only the transmission of high-frequency signals and did not change the transmission of low-frequency signals. Since radiofrequency signals are generally involved in cognitive and motor control, we investigated the possibility that increased tau protein inhibits synaptic vesicle recycling. They found that an increase in the concentration of soluble tau protein was the first to inhibit endocytosis. Furthermore, fluorescent labeling of tau protein, microtubules, and dynamin-1 revealed that the tau protein-injected presynaptic terminals had increased binding of dynamin-1, rendering it unable to play a role in endocytosis. Therefore, we synthesized a number of peptides whose amino acid sequences were partially identical to those of dynamin 1, and investigated whether these peptides could prevent the tau protein from interfering with signaling by inhibiting the binding of dynamin 1 to microtubules. As a result, among the above peptides, a peptide containing a partial sequence of the pleckstrin homology domain of dynamin 1 was able to prevent the disruption of signaling and to keep endocytosis and signaling almost normal.

Based on these new findings, the present invention provides a therapeutic agent for Alzheimer's disease characterized by containing a peptide that inhibits the binding of the dynamin-1 protein to microtubules, a therapeutic method for Alzheimer's disease characterized by administering a peptide that inhibits the binding of the dynamin-1 protein to microtubules, and a method for screening substances effective for the treatment of Alzheimer's disease. The invention is described in detail below.

### <a Therapeutic Agent for Alzheimer's Disease>

The therapeutic agent for Alzheimer's disease contains a peptide that inhibits the binding of the dynamin-1 protein to microtubules. In addition, the Alzheimer's disease therapeutic agent may contain other ingredients in addition to the above peptides to the extent that it does not impair the effect of the present invention. The therapeutic agent for Alzheimer's disease of the present invention is described in detail below.

(peptide)

Dynamin is a GTPase responsible for eukaryotic endocytosis and is a member of the dynamin family of proteins. Proteins belonging to the dynamin family play a role in many processes, including mainly the cleavage of newly formed vesicles from the plasma membrane or Golgi membrane, organelle division, cytokinesis, and microbial pathogen resistance. In mammals, three different dynamins have been identified, and important amino acid sequence differences exist in their pleckstrin homology domains. One of them, dynamin 1, is expressed in neurons and neuroendocrine cells (Nature review Molecular cell biology, 2012, 13:75-88).

Peptides which inhibit the binding of the above dynamin-1 protein to microtubules contained in the Alzheimer's disease therapeutic agent are not particularly limited as long as they inhibit the binding of the dynamin-1 protein to microtubules, but for example, a protein corresponding to the dynamin 1 protein (Human: SEQ ID NO: 1, Mouse: SEQ ID NO: 2), i.e., a portion or the whole of the dynamin 1 protein, which may or may not be completely identical to a portion or the whole of the dynamin 1 protein, e.g., having at least 80%, preferably at least 85%, more preferably at least 90%, even more preferably at least 95% sequence identity are mentioned. Among them, peptides containing the whole or partial amino acid sequences of the pleckstrin homology domain of dynamin 1 (Human: SEQ ID NO: 3, Mouse: SEQ ID NO: 4) and the proline-rich domain of dynamin 1 (Human: SEQ ID NO: 5, Mouse: SEQ ID NO: 6) are preferred, peptides containing the amino acid sequences of SEQ ID NO: 7, SEQ ID NO: 8 and SEQ ID NO: 9 are more preferred, and peptides containing the amino acid sequence of SEQ ID NO: 7 are even more preferred. The amino acid sequences of the pleckstrin homology domain and the proline-rich domain of dynamin 1 are the same in humans and mice.

Other than peptides containing the above amino acid sequence, peptides containing an amino acid sequence with one or more conservative amino acid substitutions in the above amino acid sequence are similarly preferred as peptides contained in the Alzheimer's disease therapeutic agent of the present invention. The above conservative amino acid substitutions are those in which the amino acid residues are replaced by amino acid residues with similar side chains. A family of amino acid residues with similar side chains includes: They are amino acids with basic side chains (For example, lysine, arginine, histidine), amino acids with acidic side chains (e.g. aspartic acid, glutamic acid), amino acids with uncharged polar side chains (For example, glycine, asparagine, glutamine, serine, threonine, tyrosine, cysteine), amino acids with non-polar side chains (For example, alanine, valine, leucine, isoleucine, proline, phenylalanine, methionine, tryptophan), amino acids with beta-branched side chains (For example, threonine, valine, isoleucine), and amino acids with aromatic side chains (For example, tyrosine, phenylalanine, tryptophan).

Other than the peptide containing the above amino acid sequence, peptides having at least 80% amino acid sequence identity with the above amino acid sequence, preferably at least 90% amino acid sequence identity, more preferably at least 95% amino acid sequence identity, and even more preferably at least 99% amino acid sequence identity are also listed as peptides contained in the Alzheimer's disease therapeutic agent.

The term “sequence identity” means the amount (number) of amino acid sequences (or polypeptide sequences) or polynucleotide sequences (or nucleotide sequences) that can be determined to be identical between two chains in terms of their conformity to each other between each amino acid residue or each nucleotide that makes up the chain, It means the degree of sequence correlation between two amino acid sequences or two polynucleotide sequences. The identity can be easily calculated. Many methods are known to measure the identity between two amino acid sequences or polynucleotide sequences, and the term “sequence identity” is well known to those skilled in the art.

Furthermore, the peptides contained in the therapeutic agent of Alzheimer's disease of the present invention include peptides comprising an amino acid sequence in which one or several amino acids are deleted, substituted, inserted or added in the above amino acid sequence, and having the function of inhibiting the binding of microtubules to the dynamin 1 protein. Here, “several” can be at most 10, 9, 8, 7, 6, 5, 4, 3 or 2.

Mutant DNA can be prepared by any method known to those skilled in the art, such as chemical synthesis, genetic engineering, and mutagenesis. Specifically, mutant DNA can be obtained by introducing mutations into DNA consisting of the nucleotide sequences shown in each sequence number that encode the above amino acid sequences by means of contact interaction with a mutagenic agent, ultraviolet ir-radiation, genetic engineering, or other methods. Here, site-directed mutagenesis, one of the genetic engineering methods, is useful because it is a method that can introduce a specific mutation at a specific position, as described in Sambrook, J. et al. Harbor Laboratory Press, Cold Spring Harbor, N.Y., 1989. By expressing the mutant DNA using an appropriate expression system, peptides comprising an amino acid sequence in which one or more amino acids are deleted, substituted, inserted, or added can be obtained.

The peptide contained in the Alzheimer's disease therapeutic agent of the present invention may contain an additional amino acid residue in the above amino acid sequence as long as the peptide exhibits an effect to inhibit the binding of the dynamin-1 protein to microtubules. For example, the N-terminus and/or C-terminus of the above amino acid sequence may contain at least one additional amino acid residue.

The additional amino acid residues are added, for example, for the production, purification, and stabilization of polypeptides in vivo, for coupling with other molecules, and for detection. The above additional amino acid residues may also be polypeptides with brain transit activity, amino acid motif sequences, etc.

The peptides contained in the therapeutic agent for Alzheimer's disease can be all L-amino acids, all D-amino acids, or a mixture of L-amino acids and D-amino acids, but peptides that are all L-amino acids are preferred. Peptides containing two or more asymmetric carbon atoms can be enantiomers or diastereomers of any form in any ratio.

The length of the peptides contained in the Alzheimer's disease therapeutic agent of the present invention is not particularly limited, but is preferably from 5 to 50 amino acid residues, more preferably from 8 to 4 0 amino acid residues, and most preferably 10 to 35 amino acid residues.

The peptides contained in the therapeutic agent for Alzheimer's disease of the present invention are obtained, for example, by liquid-phase peptide synthesis or solid-phase peptide synthesis according to methods known in the art.

The peptides contained in the therapeutic agent for Alzheimer's disease of the present invention may be in the form of salts and preferably contain pharmaceutically acceptable counterions (For example, chlorine, sulfuric acid, citric acid, phosphoric acid, acetic acid, sodium, potassium, calcium, magnesium). The peptide salt can be an acid addition salt or a base addition salt. Exemplary acids that can be used to form acid adducts include hydrochloric acid, sulfuric acid, nitric acid, phosphoric acid, hydrobromic acid, perchlorate, citric acid, succinic acid, maleic acid, fumaric acid, malic acid, tartaric acid, p-toluenesulfonic acid, benzenesulfonic acid, methanesulfonic acid, and trifluoroacetic acid. Exemplary bases that can be used to form base addition salts include sodium hydroxide, potassium hydroxide, alkali metal bases such as lithium hydroxide, calcium hydroxide, and alkaline earth metal salt bases such as magnesium hydroxide.

The peptide contained in the therapeutic agent for Alzheimer's disease may have some or all of the amino acid residues modified in its amino acid sequence. Such modified peptides can be prepared by any method known in the art. For example, modified peptides can be prepared by modification such as esterification, alkylation, halogenation, phosphorylation, sulfonation, amidation, etc., of functional groups in the side chains of amino acid residues constituting the peptide.

The peptides contained in the therapeutic agent for Alzheimer's disease may be modified to enhance the permeability of the blood-brain barrier.

The peptides contained in the therapeutic agent for Alzheimer's disease can, as a non-limiting example, add polypeptides, amino acid motif sequences, etc. for enhancing membrane permeability and promoting brain translocation. Non-limiting examples of polypeptides, amino acid motif sequences that can be added are described, for example, in WO 1989010134, WO 2005014625, WO 2019126240, U.S. Pat. No. 7,927,811, all of which are incorporated herein by reference. Specific examples of polypeptides and amino acid motif sequences that are added to peptides contained in the Alzheimer's disease therapeutic agent to enhance membrane permeability and promote brain translocation include peptides containing the sequence represented by SEQ ID NO: 10˜15 (Table 1), and peptides in which these polypeptides are added to peptides contained in the Alzheimer's disease therapeutic agent include peptides represented by SEQ ID NO: 16˜ 27 (Table 2).

TABLE 1

| SQID.No. | Sequence |
|---|---|
| 10 | Y G R K K R R Q R R R |
| 11 | A A V A L L P A V L L A L L A P |
| 12 | A A A A E K A A A A E K |
| 13 | R Q I K I W F Q N R R M K W K K |
| 14 | A A V L L P V L L A A P |
| 15 | R R L S Y S R R R F |

TABLE 2

| SQID.No. | Sequence |
|---|---|
| 16 | A A E K K Y M L S V D N L K Y |
| | G R K K R R Q R R R |
| 17 | A A E K K Y M L S V D N L K A |
| | A V A L L P A V L L A L L A P |
| 18 | A A E K K Y M L S V D N L K A |
| | A A A E K A A A A E K |
| 19 | A A E K K Y M L S V D N L K R |
| | Q I K I W F Q N R R M K W K K |
| 20 | A A E K K Y M L S V D N L K A |
| | A V L L P V L L A A P |
| 21 | A A E K K Y M L S V D N L K R |
| | R L S Y S R R R F |
| 22 | A A K V L N D E S K M Y L K Y |
| | G R K K R R Q R R R |
| 23 | A A K V L N D E S K M Y L K A |
| | A V A L L P A V L L A L L A P |
| 24 | A A K V L N D E S K M Y L K A |
| | A A A E K A A A A E K |
| 25 | A A K V L N D E S K M Y L K R |
| | Q I K I W F Q N R R M K W K K |
| 26 | A A K V L N D E S K M Y L K A |
| | A V L L P V L L A A P |
| 27 | A A K V L N D E S K M Y L K R |
| | R L S Y S R R R F |

The peptides contained in the therapeutic agent for Alzheimer's disease may, as a non-limiting example, add, fuse or conjugate receptors expressed at the cerebrovascular barrier, as a non-limiting example, antigen-binding fragments derived from antibodies that bind to insulin receptors, insulin-like growth factor (IGF) receptors, leptin receptors, or lipoprotein receptors, or transferrin receptors, to enhance membrane permeability and promote brain translocation by introducing modifications such as glycosylation.

The peptides contained in the Alzheimer's therapeutic agent of the present invention can also promote brain migration by packing into nanocapsule made from nanoparticles (As a non-limiting example, those described in Prog Neuropsychopharmacol Biol Psychiatry, 23,941-949, 1999, the entire contents of which are incorporated herein by reference.) that can cross the blood-brain barrier.

The peptides contained in the therapeutic agent for Alzheimer's disease of the present invention may have other substances fused, coupled or added for the purpose of improving the half-life in blood, etc. Peptides may be conjugated or linked to specific substances at the N- and/or C-termini of peptides via chemicals such as cross-linkers, or via drugs suitable for linking to the side chains of amino acids, or by synthetic chemical or genetic engineering techniques. Examples of such materials include polyalkylene glycol molecules such as polyethylene glycol (PEG); Fatty acid molecules such as hydroxyethyl starch and palmitic acid; Fc region of immunoglobulin; CH3 domain of immunoglobulin; CH4 domain of immunoglobulin; Albumin or fragments thereof; Albumin-binding peptide; Albumin-binding proteins such as streptococcal protein G; and transferrin, etc. These substances are suitably used to regulate the solubility of peptides, improve the stability of peptides by improving protease resistance, etc., and to deliver peptides to specific tissues or organs.

The therapeutic agent for Alzheimer's disease of the invention can contain the above peptides at any suitable amount. Specifically, 0.1 to 100 wt %, 1 to 99 wt %, 1 to 90 wt %, 5 to 80 wt %, 10 to 75 wt %, 15 to 50 wt % of the therapeutic agent for Alzheimer's disease.

### Other Ingredients

In addition to the above peptides, the Alzheimer's disease therapeutic agent of the present invention can contain pharmaceutically acceptable carriers, diluents and/or ex-cipients. Specifically, for example, if the Alzheimer's disease therapeutic agent of the present invention is a liquid composition, it may include a suitable solvent such as water, saline, glucose solution, or ethanol. The therapeutic agent for Alzheimer's disease of the present invention can be formulated, for example, by dissolving or suspending a fixed amount of the above peptide in a solvent. Buffers, preservatives, flavorings and/or colorants may each be included in the formulation as required. When the Alzheimer's disease therapeutic agent of the present invention is an individual composition, it may contain binders, lubricants, disintegrants, colorants, flavorings, flow accelerators, and/or melters. For example, the solid formulation may include an inert carrier such as lactose, gelatin, agar, starch, sucrose, glucose, methylcellulose, dicalcium phosphate, calcium sulfate, mannitol, sorbitol, crystalline cellulose, etc. Examples of binders include sugars such as starch, gelatin, glucose, lactose and trehalose, natural and synthetic rubbers such as cornstarch, calcium lactate, acacia, tragacanth and sodium alginate, povidone, carboxymethyl cellulose, hydroxypropyl cellulose, polyethylene glycol and wax. Examples of the lubricant include sodium oleate, sodium stearate, magnesium stearate, stearic acid, sodium stearyl fumarate, anhydrous silicic acid, talc and the like. Examples of disintegrants include starch, methylcellulose, agar, bentonite, xanthan gum, croscarmellose sodium, sodium glycolate starch, etc. Solid formulations can be enteric coated using, for example, methyl methacrylate polymers, ethyl cellulose or carnauba wax.

The therapeutic agent for Alzheimer's disease of the invention can be formulated for oral or parenteral (For example, intramuscularly or intravenously) administration. The method of administration may vary according to the condition, age, etc. of the subject. The therapeutic agent for Alzheimer's disease of the invention can be formulated as any suitable dosage form such as tablets, injectable solutions, capsules, granules, powders, syrups, suspensions, suppositories, ointments, creams, gels, patches, inhalants, etc., by techniques known in the art.

An appropriate daily dose of the Alzheimer's therapeutic agent of the invention can range from 0.005 mg/kg to 500 mg/kg body weight per day (For example, 0.005 mg/kg to 100 mg/kg, 0.005 mg/kg to 30 mg/kg, 0.005 mg/kg to 1 mg/kg, 0.01 mg/kg to 30 mg/kg, 0.01 mg/kg to 3 mg/kg, 0.01 mg/kg to 1 mg/kg, 0.02 mg/kg to 5 mg/kg, 0.02 mg/kg to 2 mg/kg, 0.02 mg/kg to 1 mg/kg) as the amount of the above peptidc.

### <a Therapeutic Agent for Alzheimer's Disease Containing an Inhibitor of Dynamin 1-Microtubules Binding>

The present invention includes an Alzheimer's disease therapeutic agent containing a binding inhibitor between dynamin 1 protein and microtubules. Based on the mechanism clarified above, the above binding inhibitor is not limited to those that inhibit the binding between dynamin 1 protein and microtubules, and includes not only the specific peptides described above but also low molecular weight compounds, medium molecular weight compounds (such as peptides other than the above), high molecular weight compounds (antibodies, etc.) are included.

As the above binding inhibitors, monoclonal antibodies against dynamin 1 protein are particularly effective. As a non-limiting example, a monoclonal antibody against a protein corresponding to the dynamin 1 protein (human: SQID No. 1, mouse: SQID No. 2), i.e., a part or the whole dynamin 1 protein. It may or may not be completely identical to a portion or the whole of the dynamin 1 protein, e.g., at least 80%, preferably at least 8.5%, more preferably at least 90%, and even more preferably at least 95% sequence identity are useful. Among them, the pleckstrin homology domain of dynamin 1 (human: SQID No. 3, mouse: SQID No. 4), the proline-rich domain of dynamin 1 (human: SQID No. 5, Mouse: SQID No. 6), and monoclonal antibodies against peptides containing all or partial amino acid sequences of SQID No. 7, SQID No. 8, and SQID No. 9 are preferred. Monoclonal antibodies to peptides containing the amino acid sequence of SQID No. 7, SQID No. 8, and SQID No. 9 are more preferred.

The antibody used in the present invention is derived from any source, which is not particularly limited, and can include an antibody preferably derived from mammals and more preferably derived from human. Monoclonal antibodies derived from mammals include an antibody produced by a hybridoma and an antibody produced by a host transformed with an expression vector comprising an antibody gene using a genetic engineering technique.

Antibody-producing hybridomas can be produced essentially by using known techniques as described below. More specifically, dynamin 1 is used as a sensitizing antigen for immunization according to a conventional immunization method. The resulting immune cells are fused with known parent cells according to a conventional cell fusion method. The fused cells are screened for a monoclonal antibody-producing cell according to a conventional screening method.

Specifically, an anti-dynamin 1 antibody may be produced as follows. For example, human dynamin 1 used as an antigen for obtaining the antibody can be obtained using dynamin 1 gene/amino acid sequence disclosed in known literatures.

The sequence of dynamin 1 gene is inserted into any known expression vector system. The resulting expression vector system is used to transform suitable host cells. Dynamin 1 protein of interest is then purified from the host cells or from the culture supernatant of the host cells using any known method. The purified dynamin 1 protein may be used as a sensitizing antigen. Dynamin 1 protein produced by chemical synthesis may be also used as a sensitizing antigen. A fusion protein between dynamin 1 protein and any other protein may be also used as a sensitizing antigen.

Mammals immunized with the sensitizing antigen are not particularly limited, but are preferably selected in consideration of compatibility with parent cells used in cell fusion. Typically, rodents such as mouse, rat, and hamster are used.

Immunization of an animal with the sensitizing antigen is performed according to any known method. For example, the immunization is generally performed by in-traperitoncally or subcutaneously injecting the sensitizing antigen into a mammal. Specifically, the sensitizing antigen is diluted with Phosphate-Buffered Saline (PBS), physiological saline, or the like to provide a suitable amount of suspension. The suspension is optionally mixed with a conventional adjuvant, such as complete Freund's adjuvant, in a suitable amount and then emulsified. The emulsified product is preferably administered to a mammal in some doses every 4 to 21 days. Suitable carriers can be also used in the immunization of the sensitizing antigen.

After the immunization and confirmation of increase in the level of the desired antibody in serum, immune cells are removed from the mammal and are submitted to cell fusion. Preferred immune cells submitted to cell fusion include particularly splenic cells.

The immune cells are fused with other parent cells. The other parent cells appropriately used include mammalian myeloma cells including a variety of known cell lines.

Cell fusion between the immune cells and myeloma cells can be performed essentially using any known method, for example, according to the method of Milstein et al. (Methods Enzymol., 73, 3-46 (1981)).

More specifically, the cell fusion is performed, for example, in a standard nutrient medium in the presence of a cell fusion-promoting agent. The cell fusion-promoting agent that may be used includes, for example, polyethylene glycol (PEG) and Sendai virus (HVJ). In addition, to increase fusion efficiency, auxiliary agents such as dimethyl sulfoxide can be added and used as desired.

The immune cells and myeloma cells are preferably used, for example, as a ratio in which the number of immune cells is 1- to 10-times more than the number of myeloma cells. Culture media that can be used in the cell fusion include, for example, RPMI1640 medium and MEM medium, which both are suitable for proliferation of the myeloma cell line, and other conventional culture media used in this type of cell culture. Moreover, a serum complement such as fetal calf serum (FCS) can be also used.

In the cell fusion, a given amount of the immune cells and myeloma cells are mixed thoroughly in the culture medium. Then, a PEG solution preheated to about 37° C., in which the PEG solution has, for example, an average molecular weight of about 1000 to 6000, is typically added in a concentration of 30 to 60% (w/v) and mixed to form fusion cells of interest (hybridomas). Subsequently, to remove cell fusing agents and other agents unfavorable for the growth of the hybridomas, the following steps can be sequentially repeated: adding a suitable culture medium, centrifuging the resulting suspension, and removing the supernatant.

The hybridomas are selected by culturing the hybridomas in a conventional selective culture medium, such as HAT culture medium (a culture medium containing hy-poxanthine, aminopterin, and thymidine). The culture in HAT culture medium is continued for a period of time sufficient to kill any cells (non-fused cells) other than the hybridomas of interest, usually for a few days to weeks. The hybridomas producing the antibody of interest are then screened for and cloned using a conventional limiting dilution method.

A method for obtaining monoclonal antibodies from the hybridomas embraces a method of culturing the hybridomas according to any conventional method and collecting the culture supernatant to obtain the monoclonal antibodies or a method of administering the hybridomas to a mammal compatible with the hybridomas, allowing the hybridomas to be proliferated, and collecting the peritoneal fluid to obtain the monoclonal antibodies. The former method is suitable for obtaining highly-pure antibodies while the latter method is suitable for high volume production of antibodies.

The monoclonal antibodies used in the present invention may be recombinant antibodies, which are produced by a gene recombination technique, which technique comprises cloning an antibody gene from a hybridoma, inserting the gene into a suitable vector, and transducing the vector into a host (see, e.g., Borrebacck C. A. K. and Larrick J. W. THERAPEUTIC MONOCLONAL ANTIBODIES, Published in the United Kingdom by MACMILLAN PUBLISHERS LTD, 1990).

Specifically, mRNA encoding the variable (V) region of the antibody of interest is isolated from cells producing the antibody, for example, hybridomas. The mRNA is isolated by preparing total RNA using any known method, such as guanidine ultracentrifugation (Chirgwin, J. M. et al., Biochemistry (1979) 18, 5294-5299) and the AGPC method (Anal. Biochem. (1987) 162, 156-159), to prepare mRNA, for example, using mRNA Purification Kit (from Pharmacia). Alternatively, mRNA can be directly prepared by using QuickPrep mRNA Purification Kit (from Pharmacia).

The obtained mRNA is used with a reverse transcriptase to synthesize cDNA of the V region of antibody. The cDNA can be synthesized by using AMV Reverse Transcriptase First-strand cDNA Synthesis Kit and the like. The cDNA can be also synthesized and amplified using 5′-RACE (Frohman, M. A. et al., Proc. Natl. Acad. Sci. USA (1988) 85, 8998-9002; Belyavsky, A. et. al., Nucleic Acid Res. (1989) 17, 2919-2932) with 5′-Ampli FINDER RACE Kit (from Clontech) and PCR. The resulting PCR products are purified to obtain the DNA fragment of interest, and the fragment is linked to a vector DNA. Furthermore, the desired recombinant vector is prepared by using the linked vector DNA to make a recombinant vector, transducing the recombinant vector into Escherichia coli cells or the like, and selecting colonies. The base sequence of the DNA of interest is confirmed using any known method, for example, the deoxy method.

Once the DNA encoding the V region of the antibody of interest is obtained, this DNA is linked to a DNA encoding the desired constant region (C region) of antibody and the linked DNA is inserted into an expression vector. Alternatively, the DNA encoding the V region of antibody may be inserted into an expression vector comprising the DNA of the C region of antibody.

For production of the antibody used in the present invention, an antibody gene is inserted into an expression vector to allow the antibody gene to be expressed under the control of an expression regulatory region, such as enhancer and promoter, as described below. The expression vector is then transformed into host cells to permit expression of the antibody.

Gene recombinant antibodies that have been artificially modified for decreasing xenogeneic antigenicity against human and the like can be used in the present invention. The gene recombinant antibodies include, for example, chimeric antibodies, humanized antibodies, and human antibodies. These modified antibodies can be produced using any known method.

Chimeric antibodies are obtained by linking the DNA encoding the V region of antibody obtained as described above to the DNA encoding the C region of human antibody, inserting the linked DNA into an expression vector, transducing the expression vector into a host, and allowing the host to produce the chimeric antibodies (see European Patent Application Publication No. EP125023, International Publication No. WO 92-19759). This known procedure can be used to obtain the chimeric antibodies useful for the present invention.

Humanized antibodies, also referred to as reshaped human antibodies or human-typed antibodies, are antibodies in which complementarity-determining region (CDR) of antibody from a nonhuman mammal, such as mouse, is grafted into the comple-mentarity-determining region of a human antibody. The general gene recombination techniques for producing humanized antibodies are known (see European Patent Application Publication No. EP125023, International Publication No. WO 92-19759).

Specifically, a DNA sequence that is designed to link CDRs of a mouse antibody to framework regions (FRs) of a human antibody is synthesized using PCR with some oligonucleotides produced to have overlapping sequences in the terminal parts of the oligonucleotides. The resulting DNA is linked to the DNA encoding the C region of a human antibody, and then inserted into an expression vector. The expression vector is transduced into a host to result in production of the antibody (see European Patent Application Publication No. EP239400, International Publication No. WO 92-19759).

The FRs of a human antibody linked to the CDRs are selected to allow the comple-mentarity-determining regions to form a functional antigen binding site. One or more amino acids in the framework region of the variable region of an antibody may be optionally substituted to allow complementarity-determining regions of a reshaped human antibody to form a functional antigen binding site as required (Sato, K. et al., Cancer Res. (1993) 53, 851-856).

Chimeric antibodies and humanized antibodies have C regions of human antibodies. The C regions of human antibodies include Cγ. For example, Cγ1, Cγ2, Cγ3, or Cγ4 can be used. The C regions of human antibodies may be also modified for improving stability of the antibody or production of the antibody.

Chimeric antibodies consist of variable regions of antibodies from nonhuman mammals and C regions of human antibodies. Humanized antibodies consist of complementarity-determining regions of antibodies from nonhuman mammals and framework regions and C regions of human antibodies. Both chimeric antibodies and humanized antibodies have low antigenicity in human bodies and therefore are useful for antibodies used in the present invention.

The methods known to provide human antibodies include, in addition to the methods as described previously, a technique of obtaining human antibodies by the panning of a human antibody library. For example, a variable region of human antibodies can be expressed in the form of single-chain antibody (scFv) on the surface of phages by phage display, and the phages that bind an antigen of interest can be selected. The selected phages are analyzed for the genes by sequencing the DNA encoding variable regions of the human antibodies that bind the antigen of interest. Once the DNA sequences of scFvs that bind the antigen are determined, expression vectors suitable for the sequences can be made. The expression vectors can be used to obtain human antibodies. These methods are well known and are described in WO 92/01047, WO 92/20791, WO 93/06213, WO 93/11236, WO 93/19172, WO 95/01438, and WO 95/15388, which can be referenced.

The antibody gene constructed as described above can be expressed using any known method, obtaining the antibody. For mammalian cells, the antibody can be expressed using a DNA that is obtained by operably linking a useful promoter usually used, the antibody gene to be expressed, and a poly A signal downstream of the 3′ end of the antibody gene, or a vector comprising the DNA. Promoters/enhancers can include, for example, human cytomegalovirus immediate early promoter/enhancer.

Other promoters/enhancers that can be used for antibody expression in the present invention include a promoter/enhancer of viruses such as retrovirus, polyomavirus, adenovirus, and simian virus 40 (SV40), and a promoter enhancer from mammalian cells such as human elongation factor 1α (HEF1α).

For example, the SV40 promoter/enhancer can be easily used according to the method of Mulligan et al. (Mulligan, R. C. et al., Nature (1979) 277, 108-114), and the HEF1αpromoter/enhancer can be easily used according to the method of Mizushima et al. (Mizushima, S. and Nagata, S. Nucleic Acids Res. (1990) 18, 5322).

For Escherichia coli, the antibody can be expressed by operably linking a useful promoter usually used, a signal sequence for antibody secretion, and the antibody gene to be expressed. The promoter can include, for example, lacZ promoter and araB promoter. The lacZ promoter may be used according to the method of Ward et al. (Ward, E. S. et al., Nature (1989) 341, 544-546; Ward, E. S. et al., FASEB J. (1992) 6, 2422-2427), and the arab promoter may be used according to the method of Better et al. (Better, M. et al., Science (1988) 240, 1041-1043).

The signal sequence for antibody secretion that is used for production in the periplasm of Escherichia coli may be pe1B signal sequence (Lei, S. P. et al., J. Bacteriol. (1987) 169, 4379-4383). The antibody produced in the periplasm is separated followed by appropriate refolding of the structure of the antibody (see, e.g., WO 96/30394).

Origins of replication that can be used include those derived from SV40, polyomavirus, adenovirus, bovine papillomavirus (BPV), and the like. Moreover, the expression vectors can comprise, as a selection marker, aminoglycoside phospho-transferase (APH) gene, thymidine kinase (TK) gene, Escherichia coli xanthine-guanine phosphoribosyltransferase (Ecogpt) gene, dihydrofolate reductase (dhfr) gene, and the like, for amplifying the gene copy number in host cell systems.

Any production system may be used for producing the antibodies used in the present invention. The production system for antibody production includes in vitro and in vivo production systems. The in vitro production system includes a production system using eukaryotic or prokaryotic cells.

The production system using eukaryotic cells includes a production system using animal, plant, or fungal cells. The animal cells known to be used include (1) mammalian cells, such as, CHO, COS, myeloma, baby hamster kidney (BHK), Hela, and Vero, (2) amphibian cells, such as Xenopus oocyte, and (3) insect cells, such as sf9, sf21, and Tn5. The plant cells known to be used include cells from Nicotiana tabacum, which may be used for callus culture. The fungal cells known to be used include yeasts, such as genus Saccharomyces including Saccharomyces cerevisiae, and filamentous bacteria, such as genus Aspergillus including Aspergillus niger. The production system using prokaryotic cells includes a production system using bacterial cells. The bacterial cells known to be used include Escherichia coli (E. coli) and Bacillus subtilis.

These cells are transformed with an antibody gene of interest, and the transformed cells can be cultured in vitro to obtain the antibody. The culture is performed according to any known method. For example, culture media that can be used include DMEM, MEM, RPMI1640, and IMDM. A serum complement such as fetal calf serum (FCS) can be also used. The cells transduced with the antibody gene may be also injected into peritoneal cavity in aminals to produce the antibody in vivo.

On the other hand, the in vivo production system includes a production system using animals and plants. The production system using animals includes a production system using mammals or insects.

The mammals that can be used include goat, pig, sheep, mouse, and cattle (Vicki Glaserm, SPECTRUM Biotechnology Applications, 1993). The insects that can be used include silkworm. The plants that can be used include, for example, tobacco.

The antibody is produced in the animals or plants into which the antibody gene has been transduced, and collected. For example, the antibody gene is interrupted by a gene encoding a protein specifically produced in milk, such as goat βcasein, to prepare a fusion gene. The DNA fragments comprising the fusion gene with the antibody gene are transferred into goat embryos. The embryos are implanted into female goats. The desired antibody is obtained from milk produced by the goats received the embryos, which are transgenic goats, or their offspring. In order to increase the amount of milk comprising the desired antibody produced by the transgenic goats, any suitable hormone may be administered to the transgenic goats (Ebert, K. M. et al., Bio/Technology (1994) 12,699-702).

Silkworms are also used to obtain the desired antibody by infecting the silkworms with a baculovirus having the antibody gene of interest inserted and collecting the fluid of the infected silkworms (Macda, S. et al., Nature (1985) 315, 592-594). In addition, when tobacco is used, the antibody gene of interest is inserted into a plant expression vector, such as pMON530, and the vector is transduced into a bacterium such as Agrobacterium tumefaciens. The bacterium is used to infect tobacco such as Nicotiana tabacum, and the desired antibody is obtained from leaves of the tobacco (Julian, K.-C. Ma et al., Eur. J. Immunol. (1994) 24, 131-138).

When an antibody is produced in the in vitro or in vivo production system as described above, host cells may be co-transformed with expression vectors into which a DNA encoding the heavy chain (H chain) or light chain (L chain) of the antibody is separately inserted or may be transformed with a single expression vector into which DNAs encoding H chain and L chain are inserted (see International Publication No. WO 94-11523).

The antibodies produced and expressed as described above can be separated from intracellular or extracellular components or hosts and purified to homogeneity. The antibodies used in the present invention can be separated and purified by affinity chromatography. Columns used in affinity chromatography include, for example, protein A columns and protein G columns. Carriers for protein A columns include, for example, Hyper D, POROS, and Sepharose F. F. Other details are not limited at all as long as the details are used for a method for separating and purifying usual proteins.

For example, the antibody used in the present invention can be separated and purified by appropriately selecting or combining a chromatography except the affinity chromatography as described above, a filter, ultrafiltration, salt precipitation, dialysis, and the like. The chromatography includes, for example, ion exchange chromatography, hydrophobic chromatography, and gel filtration. These chromatographies are applicable to high performance liquid chromatography (HPLC). Reverse phase HPLC may be also used.

The concentration of the antibody obtained above can be measured by determination of absorbance, ELISA, or the like. More specifically, in the determination of absorbance, the antibody is suitably diluted with PBS (-) followed by determination of absorbance at 280 nm to calculate the concentration of the antibody by setting the absorbance at the concentration of 1 mg/ml to 1.350 D. In ELISA, the concentration of the antibody can be measured as follows. One hundred μl of goat anti-human IgG (from TAG) diluted to 1 μg/ml with 0.1 M bicarbonate buffer (pH 9.6) is added to a 96-well plate (from Nunc) and incubated at 4° C. overnight to immobilize the antibody. After blocking, 100 μl of the antibody used in the present invention or a sample containing the antibody that is suitably diluted, or human IgG (from CAPPEL) as a reference standard is added and incubated at room temperature for an hour.

After washing, 100 μl of alkaline phosphatase-labelled human IgG diluted 5000-fold (from Bio Source) is added and incubated at room temperature for an hour. After washing, a substrate solution is added and incubated. The concentration of the antibody of interest is then calculated by determination of absorbance at 405 nm on the MICROPLATE READER Model 3550 (from Bio-Rad).

The antibody used in the present invention may be an antibody conjugated with any of various molecules including polyethylene glycol (PEG), a radioactive substance, and a toxin. Such a conjugated antibody can be obtained by chemically modifying the antibody produced as described above. The methods of modifying an antibody have been established in the art. The “antibody” in the present invention includes also the conjugated antibodies.

The antibody according to the present invention includes not only a divalent antibody as typified by IgG, but also a monovalent antibody and a polyvalent antibody as typified by IgM. The polyvalent antibody according to the present invention includes a polyvalent antibody whose antigen binding sites are all the same or a polyvalent antibody whose antigen binding sites are different in part or in whole.

The antibody according to the present invention may be a low molecular weight antibody. The low molecular weight antibody includes an antibody fragment lacking a portion of a whole antibody, such as whole IgG.

Antibody fragments can be produced by digesting an antibody with an enzyme. Enzymes known to produce antibody fragments include, for example, papain, pepsin, and plasmin. Alternatively, antibody fragments can be produced by constructing DNAs encoding the antibody fragments, inserting the DNAs into expression vectors, and then allowing the expression vectors to be expressed in suitable host cells (e.g., Co M. S. ct al., J. Immunol. (1994) 152, 2968-2976, Better M. & Horwitz A. H., Methods in Enzymology (1989) 178, 476-496, Pluckthun A. & Skerra A., Methods in Enzymology (1989) 178, 497-515, Lamoyi E., Methods in Enzymology (1986) 121, 652-663, Rousseaux J. et al., Methods in Enzymology (1986) 121, 663-669, Bird R. E. & Walker B. W., Trends Biotechnol. (1991) 9, 132-137).

Each of the enzymes for digestion cleaves an antibody at the specific position to provide antibody fragments having specific structures as described below. On the other hand, genetic engineering techniques can be used to delete any portion of an antibody:
in papain digestion: Fab;
in pepsin digestion: F(ab′)₂ or F(ab′); and
in plasmin digestion: Facb

An scFv is obtained by linking a VH and a VL of an antibody. In an scFv, the VH and VL are linked by a linker, preferably a peptide linker (Huston J. S. et al., Proc. Natl. Acad. Sci. USA (1988) 85, 5879-5883). The VH and VL in an scFv may be derived from any antibody described herein. The peptide linker linking V regions is not particularly limited. Any single-stranded peptide, for example, consisting of about 3 to 25 residues, may be used for the linker.

The V regions can be linked by, for example, PCR as described above. For the linking of V regions by PCR, a DNA encoding a full or desired partial amino acid sequence encoded by a DNA sequence encoding an H chain or the V region of an H chain of an antibody and a DNA sequence encoding an L chain or the V region of an L chain of an antibody is used as a template. Each of the DNAs encoding the V region of H chain and L chain is amplified by PCR using primers having sequences corresponding to the sequences at both ends of the DNA to be amplified. A DNA encoding a peptide linker is then prepared. The DNA encoding the peptide linker can be also synthesized by PCR. The primers used in this PCR have base sequences, which can connect to each of the amplified products of the V regions separately synthesized, previously added to the 5′ end of the primers. Next, a PCR reaction is performed using each of the DNAs of [VH DNA], [peptide linker DNA], and [VL DNA] with primers for assembly PCR. The primers for the assembly PCR are a combination of a primer that can anneal to the 5′ end of [VH DNA] and a primer that can anneal to the 3′ end of [VL DNA]. In other words, the primers for the assembly PCR comprise a set of primers that can be used to amplify a DNA encoding the full sequence of the scFv to be synthesized. The [peptide linker DNA] has a base sequence previously added, which can connect to each of the DNAs of the V regions. The primers for the assembly PCR are used to link these DNAs, eventually producing the full-length scFv as an amplified product. Once the DNA encoding the scFv is produced, an expression vector comprising the DNA and recombinant cells transformed with the expression vector can be obtained using any conventional method. The scFv can be also obtained by culturing the resulting recombinant cells and allowing the cells to express the DNA encoding the scFv.

Diabody refers to a bivalent low molecular weight antibody constructed by gene fusion (Holliger P. ct al., Proc. Natl. Acad. Sci. USA (1993) 90, 6444-6448, EP 404097, WO 93/11161). Diabody is a dimer composed of two polypeptide chains. Generally, each of the polypeptide chains composing the dimer is linked by a linker between VL and VH in a single chain. The linker for the polypeptide chains in the diabody typically is too short to allow the VL and VH on the same chain to associate with each other. Specifically, amino acid residues composing the linker has preferably 2 to 12 residues, more preferably 3 to 10 residues, and particularly about 5 residues. Therefore, the VL and VH encoded in a single polypeptide chain are not able to form an scFv and therefore two separate polypeptide chains result in dimerization to form two Fvs. Consequently, the diabody has two antigen binding sites.

An sc (Fv) 2 is a single-stranded low molecular weight antibody, in which two VHs and two VLs are linked by linkers (Hudson P. J. & Kortt A. A., J. Immunol. Methods (1999) 231, 177-189). An sc (Fv) 2 can be produced, for example, by linking two scFvs by a linker. Alternatively, an sc (Fv) 2 can be also produced by linking two VHs and two VLs by linkers, starting from the N-terminus of the single-stranded polypeptide, in the order as described below:

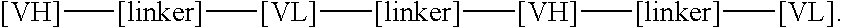

It is noted that the order of two VHs and two VLs is not particularly limited to the order as described above and any order is acceptable. For example, the orders as described below can be also included.

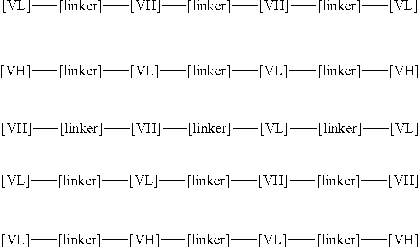

A plurality of the linkers may be the same or different type.

The linkers that can be used to link variable regions of an antibody include any peptide linker that can be incorporated by genetic engineering or a synthetic compound linker (e.g., the linker disclosed in Protein Engineering (1996) 9, 299-305). Peptide linkers are preferred in the present invention. The length of peptide linkers is not particularly limited and can be appropriately selected by those skilled in the art according to any purpose. Generally, amino acid residues composing a peptide linker have 1 to 100 amino acids, preferably 3 to 50 amino acids, more preferably 5 to 30 amino acids, particularly preferably 12 to 18 amino acids (e.g., 15 amino acids). An amino acid sequence composing a peptide linker can be any sequence unless the sequence inhibits the binding effect of scFv.

Alternatively, synthetic compound linkers (chemical cross-linkers) can be also used to link V regions. Cross-linkers that are commonly used for cross-linking of peptide compounds can be used in the present invention. The cross-linkers that can be used include, for example, N-hydroxysuccinimide (NHS), disuccinimidyl suberate (DSS), bis(sulfosuccinimidyl) suberate (BS3), dithiobis(succinimidyl propionate) (DSP), dithiobis(sulfosuccinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis[2-(succinimide oxycarbonyloxy)ethyl]sulfone (BSOCOES), and bis[2-(sulfosuccinimide oxycarbonyloxy)ethyl]sulfone (sulfo-BSOCOES).

<A Monoclonal Antibody that Inhibits Microtubule-Dynamin 1 Binding>

The present invention also includes monoclonal antibodies that inhibit the binding of microtubules to dynamin 1 protein. Monoclonal antibodies against dynamin 1 protein are effective as monoclonal antibodies of the present invention. As a non-limiting example, a monoclonal antibody against a protein corresponding to the dynamin 1 protein (human: SQID No. 1, mouse: SQID No. 2), i.e., a part or the whole dynamin 1 protein. It may or may not be completely identical to a portion or the whole of the dynamin 1 protein, e.g., at least 80%, preferably at least, preferably at least 8.5%, more preferably at least 90%, and even more preferably at least 95% sequence identity are useful. Among them, the pleckstrin homology domain of dynamin 1 (human: SQID No. 3, mouse: SQID No. 4), the proline-rich domain of dynamin 1 (human: SQID No. 5, Mouse: SQID No. 6), and monoclonal antibodies against peptides containing all or partial amino acid sequences of SQID No. 7, SQID No. 8, and SQID No. 9 are preferred. Monoclonal antibodies to peptides containing the amino acid sequence of SQID No. 7, SQID No. 8, and SQID No. 9 are more preferred.

For the method of production of the monoclonal antibody and other detailed description, the description in the section “A therapeutic agent for Alzheimer's disease containing an inhibitor of dynamin 1-microtubules binding” can be applied as is.

### <A Method for Treating Alzheimer's Disease>

The method for treating Alzheimer's disease of the present invention is characterized by administering to a patient a peptide that inhibits the binding of the dynamin-1 protein to microtubules. According to the method for treating Alzheimer's disease, the binding of dynamin-1 to microtubules can be inhibited, thereby preventing the interference of signal transduction by the tau protein by administering a peptide to a patient that inhibits the binding of the dynamin-1 protein to microtubules. As a result, endocytosis and signaling can be kept nearly normal, which can improve and treat Alzheimer's disease. The method for treating Alzheimer's disease of the present invention can also be described as a method of administering the therapeutic agent for Alzheimer's disease of the present invention to a patient.

Peptides that inhibit the binding of the dynamin 1 protein to microtubules are not particularly limited as long as they inhibit the binding of the dynamin 1 protein to microtubules, but for example, peptides whose amino acid sequence partially matches that of dynamin 1 can be cited. Among them, peptides containing the partial amino acid sequence of the pleckstrin homology domain of dynamin 1 and the partial amino acid sequence of the proline-rich domain of dynamin 1 are preferred, peptides containing the amino acid sequence of SEQ ID NO: 7, SEQ ID NO: 8 or SEQ ID NO: 9 are more preferred, and peptides containing the amino acid sequence of SEQ ID NO: 7 are even more preferred. In addition, it is also preferable to add polypeptides, amino acid motif sequences, etc. to the above peptides to enhance membrane permeability and promote brain migration. Specific examples of the polypeptides and the amino acid motif sequences for enhancing membrane permeability and promoting brain migration include peptides containing sequences represented by SEQ ID NO: 10-15. In addition, the peptide represented by SEQ ID NO: 16˜ 27 is given as a specific example of the peptide to which a polypeptide for enhancing membrane permeability and promoting brain translocation, the amino acid motif sequence, etc. are added.

For peptides that inhibit the binding of dynamin-1 protein to microtubules, the description in a therapeutic agent for Alzheimer's disease can be applied as it is.

Appropriate daily doses of the above peptides in the Alzheimer's disease treatment method of the present invention can range from 0.005 mg/kg to 500 mg/kg body weight per day (For example, 0.005 mg/kg to 100 mg/kg, 0.005 mg/kg to 30 mg/kg, 0.005 mg/kg to 1 mg/kg, 0.01 mg/kg to 30 mg/kg, 0.01 mg/kg to 3 mg/kg, 0.01 mg/kg to 1 mg/kg, 0.02 mg/kg to 5 mg/kg, 0.02 mg/kg to 2 mg/kg, 0.02 mg/kg to 1 mg/kg).

### <a Method for Screening Substances Effective for the Treatment of Alzheimer's Disease>

The method for screening substances effective for the treatment of Alzheimer's disease is characterized by including a step of measuring the inhibitory activity of the test substance in binding the dynamin-1 protein to microtubules.

Specifically, the screening method is characterized by measuring the amount of binding between dynamin-1 protein and microtubules (tubulin) in the presence and absence of the test substance, and judging the presence or absence of binding inhibitory activity of the test substance from the ratio of the amount of binding. Such screening methods of the present invention preferably include the following steps 1)-3).
1) In the binding test of dynamin-1 protein and microtubules (tubulin), the process of measuring the amount of binding of both when the test substance is added
2) The process of measuring the amount of binding between the two when the test substance is not added
3) The process of comparing the measured values from step 1) above with the measured values from step 2) above.

In step 1) of the screening method of the present invention, the test substance is first brought into contact with the dynamin-1 protein and/or microtubules (tubulin) by adding the test substance in a binding test.

Dynamin 1 protein used in the screening method of the present invention can be prepared by E. coli, insect cells, wheat germ cell-free expression system, etc. In addition, the species of the dynamin-1 protein can be selected according to the target of the therapeutic agent for Alzheimer's disease, but it is preferred to be mammalian and human. In addition, microtubules can be prepared by polymerizing tubulin purified from the brains of mammals such as pigs. The dynamin-1 protein and microtubules (tubulin) used in the screening method of the present invention include, but are not limited to, mutants, alleles, variants, homologs, partial peptides, fusion proteins with other proteins, and those labeled by tags.

The species from which the dynamin-1 protein and microtubules (tubulin) used in the screening method of the present invention are derived are not limited to specific species. Examples include humans, monkeys, mice, rats, guinea pigs, pigs, cattle, yeasts and insects.

The state of the dynamin-1 protein and microtubules (tubulin) used in the screening method of the present invention is not particularly limited and may be, for example, purified, expressed in cells, expressed in cell extracts, etc.

The test substance in the present invention is not particularly limited and examples of the substances include a single substance such as a natural compound, an organic compound, an inorganic compound, a nucleic acid, a protein (including an antibody), a peptide, etc.; expression products of compound, nucleic acid, peptide and gene libraries; cell extracts, cell culture supernatants, fermented microbial products, marine biological extracts, plant extracts, prokaryotic cell extracts, eukaryotic single-cell extracts or extracts of animal cells. The test substance in the present invention may be a mixture of these substances. These test substances can also be labeled and used as needed. Radiolabeling, fluorescent labeling and the like can be cited as labels.

In the present invention, the contact is made according to the state of the dynamin-1 protein and microtubules (tubulin). For example, if the dynamin-1 protein and microtubules (tubulin) are purified, this can be done by adding the test substance to the purified preparation. In addition, the test substance can be added to the cell culture medium or the cell extract, or directly administered to experimental animals, if the test substance is expressed in the cells or in the cell extract, respectively. If the test substance is a protein, it can also be done, for example, by introducing a vector containing DNA encoding the above protein into cells expressing the dynamin-1 protein and microtubules (tubulin), or by adding the above vector to cell extracts expressing the dynamin-1 protein and microtubules (tubulin).

In the present invention, the test substance may be brought into contact with the dynamin-1 protein and microtubules (tubulin) by adding the test substance to a sample containing the dynamin-1 protein and microtubules (tubulin). The test substance may also be added to a sample containing either the dynamin-1 protein or microtubules (tubulin), followed by the addition of the other that did not contain the test substance to bring them into contact with the test substance.

In the present invention, the amount of binding between dynamin 1 protein and microtubule (tubulin) is then measured. In step 2) of the screening method of the present invention, the amount of binding between dynamin 1 protein and microtubule (tubulin) is measured when the test substance is not added to the sample containing dynamin 1 protein and microtubule (tubulin). Specifically, the binding amounts of dynamin 1 protein and microtubule (tubulin) are measured when the test substance is added (contacted) and when the test substance is not added (not contacted), and the values are compared in step 3) of the screening method. If the amount of binding of dynamin 1 protein to microtubule (tubulin) is reduced when the test substance is added compared to the case in which the test substance is not added, it can be judged that the test substance is effective in inhibiting the binding of dynamin 1 protein and microtubule (tubulin).

As a method for measuring the amount of binding between dynamin-1 protein and microtubules (tubulin), for example, a microtubule co-precipitation assay can be used. In microtubule co-precipitation assays, the dynamin-1 protein that binds to microtubules pellets together with microtubules during centrifugation. The presence of a test substance that inhibits the binding of the dynamin-1 protein to microtubules decreases the amount of the dynamin-1 protein that pellets together, and the extent of this decrease can be used to determine the binding inhibition effect.

### EXAMPLES

Hereinafter, the present invention will be described in more detail with reference to examples, but the present invention is not limited by these examples.

### <Materials and Methods>

### Animals

All experiments were performed in accordance with the guidelines of the Physiological Society of Japan and animal experiment regulations at Okinawa Institute of Science and Technology Graduate University.

### Recombinant Human Tau Preparation

Human tau (h-tau) lacking the MT-binding domain (amino acid 244 to 367, del-MTBD) were produced by site-directed mutagenesis as previously reported (40). Wild-type (WT) and del-MTBD mutant h-tau of ON4R isoform were expressed in E. coli. (BL21/DE3) and purified as described previously (73) with minor modifications. Briefly, harvested bacteria expressing recombinant tau were lysed in homogenization buffer (50 mM PIPES, 1 mM EGTA, 1 mM DTT, 0.5 mM PMSF, and 5 μg/ml Leupeptin, pH6.4), sonicated and centrifuged at 27,000×g for 15 min. Supernatants were charged onto phosphocellulose column (P11, Whatman). After washing with homogenization buffer containing 0.1 M NaCl, h-tau-containing fractions were eluted by the buffer containing 0.3 M NaCl. Subsequently, the proteins were precipitated by 50% saturated ammonium sulfate and re-solubilized in homogenization buffer containing 0.5 M NaCl and 1% 2-mercaptoethanol. After incubation at 100° C. for 5 min, heat stable (soluble) fractions were obtained by centrifugation at 21,900×g, and fractionated by reverse phase high-performance liquid chromatography (RP-HPLC) using Cosmosyl Protein-R (Nacalai tesque Inc.). Aliquots of h-tau containing fractions were lyophilized and stored at −80° C. Purified h-tau proteins were quantified by SDS-PAGE followed by Coomassie Brilliant Blue staining.

### Purification of Recombinant Human Dynamin1 Protein

His-tagged human dynamin 1 was expressed using the Bac-to-Bac baculovirus expression system (Thermo Fisher Scientific, Waltham, MA, USA) and purified as described previously (74). The purified dynamin solutions were concentrated using Centriplus YM50 (cat #4310; Merck-Millipore, Darmstadt, Germany).

### Microtubule Polymerization Assay

Effects of tau and nocodazole on MT polymerization were tested using a Tubulin Polymerization Assay (Cytoskeleton Inc., Denver, CO). Briefly, purified WT or del-MTBD mutant h-tau (10 μM) were mixed with porcine tubulin (20 μM) in an assembly buffer at 37° C. Nocodazole was added to the mixture at 0 min of incubation. MT polymerization was fluorometrically assayed (excitation at 360 nm, emission at 465 nm) using Infinit F-200 Microplate Reader (TECAN, Mannedorf/Switzerland) at 1 min intervals for 30 min. After incubation, resultant solutions were subjected to contrifugation at 100,000 xg for 15 min at 20° C. Supernatants (free tubulin fraction) and pellets (microtubule fraction) were subjected to SDS-PAGE to quantify the amount of tubulin assembled into MTs.

### Peptide Synthesis and LC-MS/MS Analysis

The peptides were synthesized through conventional 9-fluorenylmethyloxycarbonyl (Fmoc) solid-phase peptide synthesis (SPPS), onto preloaded Fmoc-alanine TCP-resins (Intavis Bioanalytical Instruments) using automated peptide synthesizer ResPep SL (Intavis Bioanalytical Instruments). All Fmoc-amino acids were purchased from Watanabe Chemical Industries and prepared at 0.5 M in N-methyl pyrrolidone (NMP, Wako Pure Chemical Industries). After synthesis, peptides were cleaved with (v/v/v) 92.5% TFA, 5% TIPS and 2.5% water for 2 h, precipitated using t-butyl-methyl-ether at −30° C., pelleted and resuspended in water before lyophilization (EYELA FDS-1000) overnight. All synthesized peptides' purity and sequence were then confirmed by LC-MS/MS using a Q-Exactive Plus Orbitrap hybrid mass spectrometer (Thermo Scientific) equipped with Ultimate 3000 nano-HPLC system (Dionex), HTC-PAL au-tosampler (CTC Analytics), and nanoelectrospray ion source.

### MT-Dynamin Binding Assay

Microtubule binding assay was performed using Microtubule Binding Protein Spin Down Assay Kit (cat #BK029, Cytoskeleton Inc., Denver, CO, USA). Briefly, 20 μl of 5 mg/ml tubulin in general tubulin buffer (GTB; 80 mM PIPES pH7.0, 2 mM MgCl₂, 0.5 mM EGTA) supplemented with 1 mM GTP were polymerized by adding 2 μl of cushion buffer (80 mM PIPES pH7.0, 1 mM MgCl₂, 1 mM EGTA, 60% glycerol), and incubated at 35° C. for 20 min. Microtubules were stabilized with 20 μM Taxol. Taxol-stabilized microtubules (2.5 μM) and dynamin 1 (1 M) were incubated in GTB with or without 1 mM peptide at room temperature for 30 min. After incubation, the 50 μl of mixture was loaded on top of 100 μl cushion buffer supplemented with 20 μM Taxol, and then centrifuged at 100,000×g for 40 min at room temperature. After the ultracentrifugation, 50 μl of supernatant was taken and mixed with 10 μl of 5×sample buffer. The resultant pellet was resuspended with 50 μl of 1×sample buffer. Twenty μl of each sample was analyzed by SDS-PAGE and stained with SYPRO Orange. Protein bands were visualized using FLA-3000 (FUJIFILM Co. LTD, Tokyo, Japan).

### Immunocytochemical Analysis

The following primary antibodies were used: anti-β3-tubulin (Synaptic System, #302304), anti-human Tau (BioLegend, #806501), anti-dynamin (Invitrogen, PA1-660). Secondary antibodies were goat IgG conjugated with Alexa Fluor 488, 568, or 647 (Thermo Fisher Scientific). Acute brainstem slices (175 μm in thickness, see below) were fixed with 4% paraformaldehyde in PBS for 30 min at 37° C. and overnight at 4° C. On the following day, slices were rinsed three times in PBS, permeabilized in PBS containing 0.5% Triton X-100 (Tx-100; Nacalai Tesque) for 30 min and blocked in PBS containing 3% bovine serum albumin (BSA; Sigma-Aldrich) and 0.05% Tx-100 for 45 min. Slices were incubated overnight at 4° C. with primary antibody diluted in PBS 0.05% Tx-100, 0.3% BSA. On the next day, slices were rinsed three times with PBS containing 0.05% Tx-100 for 10 min and incubated with corresponding secondary antibody diluted in PBS 0.05% Tx-100, 0.3% BSA for 1 h at room temperature (RT). Slices were further rinsed three times in PBS 0.05% Tx-100 for 10 min and finally washed in PBS for another 10 min. Finally, slices were mounted on glass slides (Matsunami) using liquid mounting medium (Ibidi) and sealed using nail polish. Confocal images were acquired on laser scanning microscopes (LSM780 or LSM900, Carl Zeiss) equipped with a Plan-apochromat 63x oil-immersion objective (1.4 NA) and 488, 561, and 633 nm excitation laser lines. For quantifying fluorescence intensity levels, the region of interest was delimited around calyceal terminals, and background fluorescence was subtracted using ImageJ software.

### Purification of GST-proteins

The cDNA encoding PH domain (521-618 amino acids) of human dynamin 1 (NM_004408.4) (75) were prepared by PCR and subcloned into the plasmid pGEX-6P vector. The resulting plasmid was transformed into bacterial BL21 (DE3) pLysS strain for protein expression. The expression of GST-fusion proteins was induced by 0.1 mM isopropyl-1-thio-D-galactopyranoside (IPTG) at 37° C. for 3-6 h in LB media supplemented with 100 μg/ml ampicillin at Aww=0.8. GST-fusion proteins were then purified as described (76). The nucleotide sequences of the constructs using in this study were verified with DNA sequence analysis. All the purified protein solutions (1-3 mg/ml) were stored at −80° C. and thawed at 37° C. before use.

Microscopic observation of microtubule and GST-PH protein GST or GST-PH was labeled using HiLyte Fluor-555 labeling kit according to manufacture's manual (cat #LK14, Dojindo Co. LTD, Kumamoto, Japan). HiLyte Fluor-555 labeled GST or GST-PH was mixed with non-labelled each protein at the ratio of 1:1.2. Flutax 1-stabilized microtubules (4.1 μM) and fluorescent GST or GST-PH at 11 μM were mixed in GTB containing 2 μM Flutax 1 at 37° C. for 60 min. Eight μl of the mixture was spotted on the slide glass and mounted with Fluoromount (cat #K024, Di-agonistic BioSystems, CA, USA). Samples were examined using a spinning disc confocal microscope system (X-Light Confocal Imager; CREST OPTICS S. P. A., Rome, Italy) combined with an inverted microscope (IX-71; Olympus Optical Co., Ltd., Tokyo, Japan) and an iXon+camera (Oxford Instruments, Oxfordshire, UK). The confocal system was controlled by MetaMorph software (Molecular Devices, Sunnyvale, CA, USA). When necessary, images were processed using Adobe Photoshop CS3 or Illustrator CS3 software. For electron microscopic observation, samples were submitted to negative staining for imaging with a transmission electron microscope (TEM) (H-7650, Hitachi High-Tech Corp., Tokyo, Japan) at 120 kV.

### Slice Electrophysiology

After killing C57BL/6N mice of either sex (postnatal day 13-15) by decapitation under isoflurane anesthesia, brainstems were isolated and transverse slices (175 μm thick) containing the medial nucleus of the trapezoid body (MNTB) were cut using a vibratome (VT1200S, Leica) in ice-cold artificial cerebrospinal fluid (aCSF, see below) with reduced Ca²⁺ (0.1 mM) and increased Mg²⁺ (3 mM) concentrations or sucrose-based aCSF (NaCl was replaced to 300 mM sucrose, concentrations of CaCl₂) and MgCl₂ was 0.1 mM and 6 mM, respectively). Slices were incubated for 1 h at 36-37° C. in standard aCSF containing (in mM); 125 NaCl, 2.5 KCl, 26 NaHCO₃, 1.25 NaHPO₄, 2 CaCl₂), 1 MgCl₂, 10 glucose, 3 myo-inositol, 2 sodium pyruvate, and 0.5 sodium ascorbate (pH 7.4 when bubbled with 95% O2 and 5% C₀2, 310-320 mOsm), and maintained thereafter at room temperature (RT, 24-28° C.).

Whole-cell recordings were made using a patch-clamp amplifier (Multiclamp 700A, Molecular Devices, USA for pair recordings and EPC-10 USB, HEKA Elektronik, Germany for presynaptic capacitance measurements) from the calyx of Held presynaptic terminals and postsynaptic MNTB principal neurons visually identified with a 60× or 40× water immersion objective (LUMPlanFL, Olympus) attached to an upright microscope (Axioskop2, Carl Zeiss, or BX51WI, Olympus, Japan). Data were acquired at a sampling rate of 50 kHz using pClamp (for Multiclamp 700A) or Patchmaster software (for EPC-10 USB) after online filtering at 5 kHz. The presynaptic pipette was pulled for the resistance of 7-10 MSΩ and had a series resistance of 14-20 MS2, which was compensated by 70% for its final value to be 7 MQ2. Resistance of the postsynaptic pipette was 5-7 MS2, and its series resistance was 10-25 MS2, which was compensated by up to 75% to a final value of 7 MS2. The aCSF routinely contained picrotoxin (10 μM) and strychnine hydrochloride (0.5 μM) to block GABA receptors and glycine receptors, respectively. Postsynaptic pipette solution contained (in mM): 130 CsCl, 5 EGTA, 1 MgCl₂, 5 QX314-C1, 10 HEPES (adjusted to pH 7.3-7.4 with CsOH). The presynaptic pipette solution contained (in mM); 105 K methanesulfonate, 30 KCl, 40 HEPES, 0.5 EGTA, 1 MgCl₂, 12 phosphocreatine (Na salt), 3 ATP (Mg salt), 0.3 GTP (Na salt) (pH 7.3-7.4 adjusted with KOH, 315-320 mOsm).

In simultaneous presynaptic and postsynaptic whole-cell recordings, postsynaptic MNTB neurons were voltage-clamped at the holding potential of −70 mV, and EPSCs were evoked, at 0.1 Hz or 1 Hz, by action potentials elicited by a depolarizing current (1 ms) injected in calyceal terminals. For intra-terminal loading of taxol (1 μM), it was diluted in presynaptic pipette solution from 5 mM DMSO stock for final DMSO con-centration to be 0.02%. Likewise, nocodazole (20 μM, 0.1% DMSO) was included in presynaptic pipette solution. Presynaptic pipette solutions in nocodazole controls contained 0.1% DMSO. In simultaneous pre- and postsynaptic recordings, WT h-tau, del-MTBD tau, taxol or synthetic peptides were loaded in calyceal terminals using the pipette perfusion technique (42, 77). Briefly, a fine superfusion tube composed of plastic and glass tubes was installed in a presynaptic patch pipette. After back-filling the tube with pipette solutions containing proteins and/or peptides, it was inserted into a patch pipette with its tip 500-600 μm behind the tip of presynaptic patch pipette. After recording baseline EPSCs, the tube solution was delivered into presynaptic patch pipette with a positive pressure (8-10 psi) applied using a pico-pump.

Membrane capacitance (Cₘ) measurements were made from calyx of Held presynaptic terminals in the whole-cell configuration at RT (47, 49). Calyceal terminals were voltage-clamped at a holding potential of −80 mV, and a sinusoidal voltage command (1 kHz, 60 mV in peak-to-peak amplitude) was applied. To isolate presynaptic voltage-gated Ca²⁺ currents (I_{ca}), the aCSF contained 10 mM tetracthy-lammonium chloride, 0.5 mM 4-aminopyridine, 1 μM tetrodotoxin, 10 μM bicuculline methiodide and 0.5 μM strychnine hydrochloride. The presynaptic pipette solution contained (in mM): 125 Cs methanesulfonate, 30 CsCl, 10 HEPES, 0.5 EGTA, 12 disodium phosphocreatine, 3 MgATP, 1 MgCl₂, 0.3 Na₂GTP (pH 7.3 adjusted with CsOH, 315-320 mOsm). Tau or synthetic peptides were dissolved in pipette solution and backfilled into the pipette briefly after loading the tau-free pipette solution from the pipette tip. Care was taken to maintain series resistance <16 MSΩ to allow dialysis of the terminal with pipette solution. Recording pipette tips were coated with dental wax to minimize stray capacitance (4-5 pF). Single square pulse (−80 to 10 mV, 20 ms duration) was used to induce presynaptic I_{Ca}. In these experiments, exocytic capacitance change (ΔCm) represents ˜5 times larger number of SVs (estimated from ΔCm divided by Cm of single SV) than that in the immediately releasable pool by presynaptic action potentials (estimated by the size of maximally evoked EPSCs divided by the size of miniature EPSCs). Membrane capacitance changes within 450 ms of square-pulse stimulation were excluded from analysis to avoid contamination by conductance-dependent capacitance artifacts (49). To avoid the influence of capacitance drift on baseline, we removed data when the baseline drift measured 0-10 s before stimulation was over 5 fFs 1. When the drift was 1-5 fFs 1, we subtracted a linear regression line of the baseline from the data for the baseline correction. The endocytic rate was calculated from the slope of the normalized C. changes during the initial 10 s after the stimulation.

### Data Analysis and Statistics

Data were analyzed using IGOR Pro 6 (WeveMatrics), Excel 2016 (Microsoft), and StatPlus (AnalystSoft Inc) and KaleidaGraph for Macintosh, version 4.1 (Synergy Software Inc., Essex Junction, VT, USA). All values are given as mean+S.E.M. Differences were considered statistically significant at p<0.05 in paired or unpaired t-tests, one-way ANOVA with Scheffe post-hoc test and repeated-measures two-way ANOVA with post-hoc Scheffe test.

### <Results>

Intra-terminal loading of WT h-tau impairs excitatory synaptic transmission

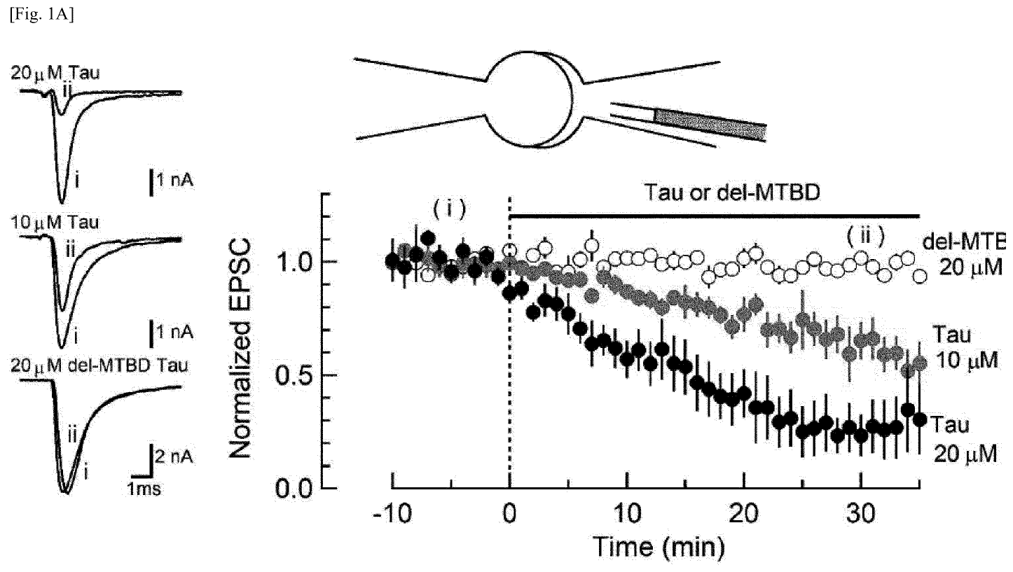

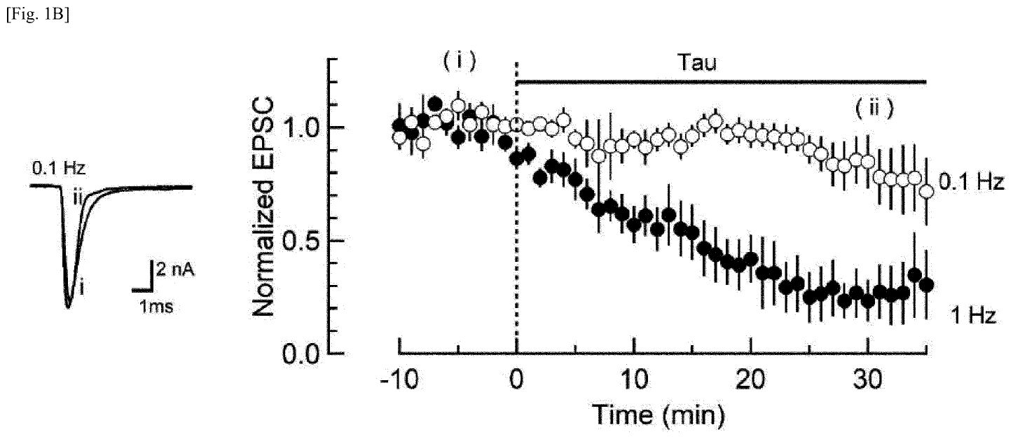

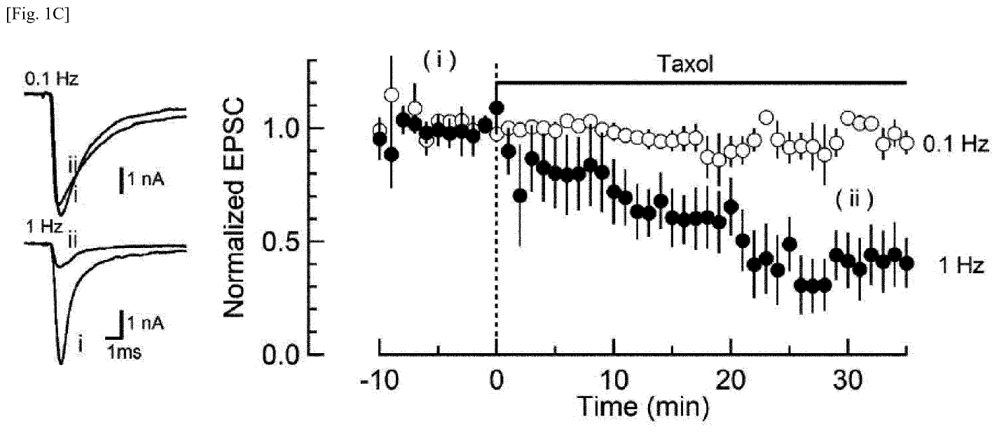

To address whether elevation of soluble h-tau in presynaptic terminals can affect synaptic transmission, we purified WT recombinant h-tau (ON4R) and its deletion mutant (del-MTBD) lacking the MT binding site (244Gln-367Gly) (FIG. 1-figure supplement 1A), obtained using an E. coli expression system (40). These recombinant h-tau proteins are highly soluble at room temperature without any sign of granulation (41). In simultaneous pre- and postsynaptic recording at the calyx of Held in mouse brainstem slices, we recorded EPSCs evoked at 1 Hz by presynaptic action potentials (FIG. 1). After confirming stable EPSC amplitude for 10 min, we injected a large volume of internal solution containing WT h-tau (20 μM) from an installed fine tube to a presynaptic whole-cell pipette to replace most of pipette solution and allow h-tau to diffuse into a presynaptic terminal (illustration in FIG. 1A) (42, 43). After loading h-tau (20 μM), the amplitude of glutamatergic EPSCs gradually declined and reached 23±9% in 30 min (FIG. 1A, p<0.01, paired t-test, n=6 synapses in 6 slices). WT h-tau loaded at a lower concentration (10 μM) caused a slower EPSC rundown to 65±5% in 30 min (p<0.01, n=5 synapses in 5 slices). Del-MTBD (20 μM), lacking tubulin polymerization capability (FIG. 1-figure supplement 1B), likewise loaded had no effect on EPSC amplitude (FIG. 1A). Since h-tau concentrations in presynaptic terminals are equilibrated with those in a presynaptic whole-cell pipette with a much greater volume than terminals (44), these results suggest that WT h-tau>10 μM can significantly impair excitatory synaptic transmission.

The inhibitory effect of WT h-tau on EPSCs was apparently frequency-dependent. When evoked at 0.1 Hz, WT h-tau (20 μM) caused only a minor reduction of EPSC amplitude (to 85±12%, 30 minutes after loading, p=0.21, n=5; FIG. 1B). Since taxol shares a common binding site of MTs with tau (45) and assembles tubulins into MTs (FIG. 1-figure supplement 1B), we tested the effect of taxol (1 μM) on EPSCs (FIG. 1C). Like h-tau, taxol caused a significant rundown of EPSCs evoked at 1 Hz (to 41±12 at 30 min, n=5, p<0.05), but not those evoked at 0.1 Hz (104±3.0%, n=4, p=0.60). These results together suggest that MTs newly assembled in presynaptic terminals by WT h-tau or taxol cause activity-dependent rundown of excitatory synaptic transmission.

WT h-Tau Primarily Inhibits SV Endocytosis and Secondarily Exocytosis

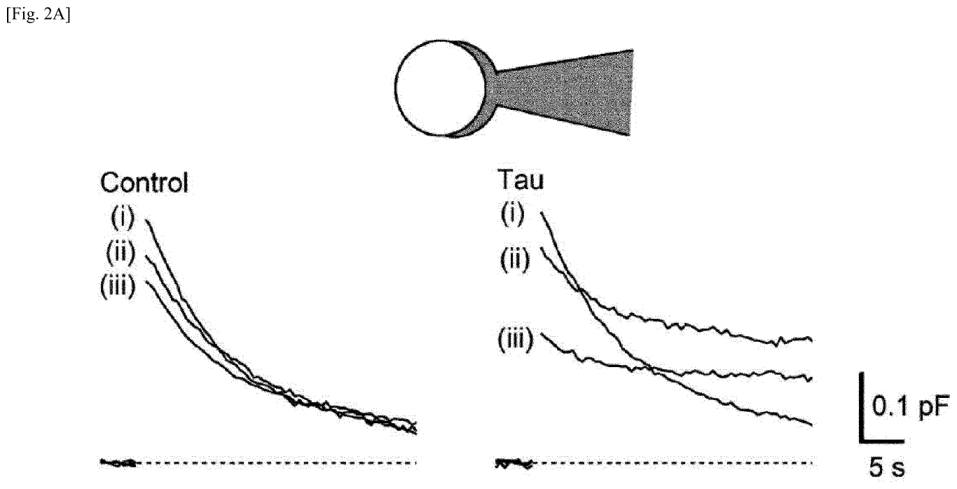

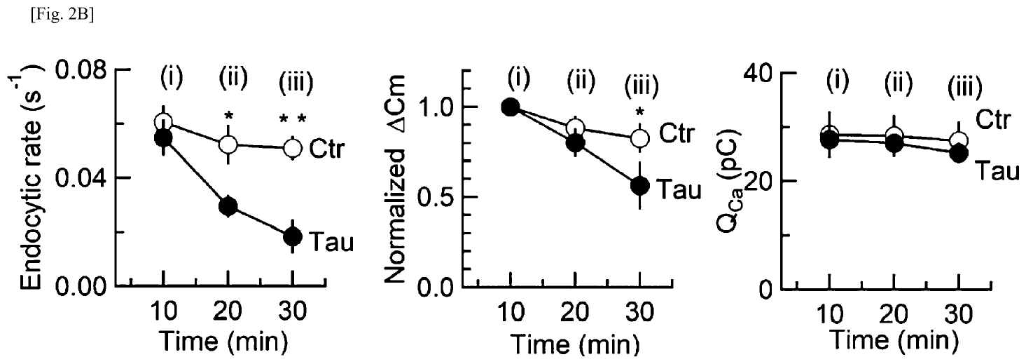

To determine the primary target of h-tau causing synaptic dysfunction, we performed membrane capacitance measurements at the calyx of Held (46-49). Since stray capacitance of perfusion pipettes prevents capacitance measurements, we backfilled h-tau into a conventional patch pipette after preloading normal internal solution only at its tip to secure GS2 scal formation. This caused substantial and variable delays of the intra-terminal diffusion, so no clear effect could be seen more than 10 minutes after whole-cell patch membrane was ruptured. 20 minutes after whole-cell patch-loading of WT h-tau (20 μM), endocytic capacitance showed a significant slowing (FIG. 2), whereas exocytic capacitance magnitude (AC . . . ) or charge of Ca²⁺ currents (Q_{Ca}) induced by a depolarizing pulse was not different from controls without h-tau loading. 30 minutes after loading h-tau, the endocytic capacitance change became further slowed (p<0.01), and exocytic AC, eventually showed a significant reduction (p<0.05, n=5) without a change in Q_{Ca}. These results suggest that the primary target of h-tau toxicity is synaptic vesicle (SV) endocytosis. Endocytic block inhibits recycling re-plenishment of SVs via recycling, thereby reducing the exocytic release of neurotransmitter as a secondary effect.

Inhibition of SV endocytosis and synaptic transmission by WT h-tau requires de novo MT assembly

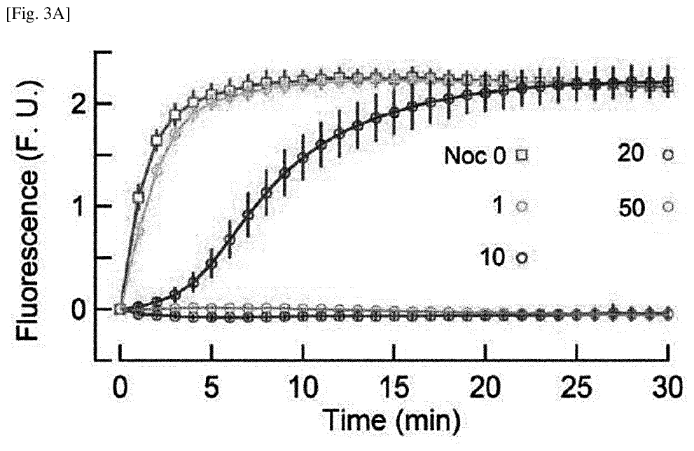

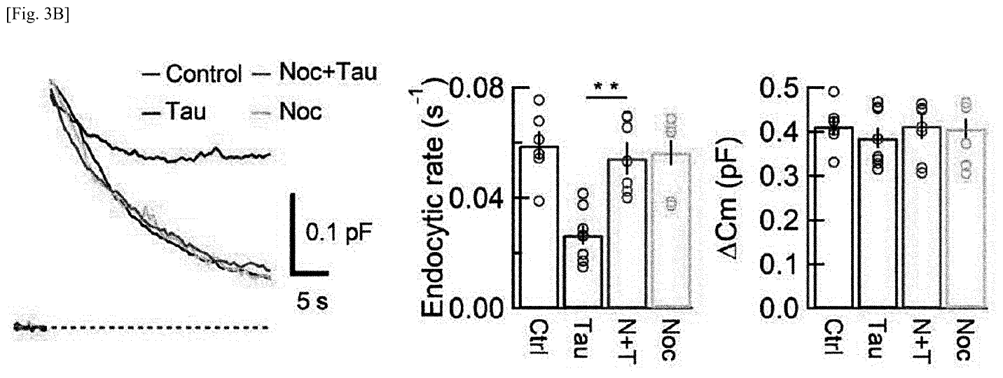

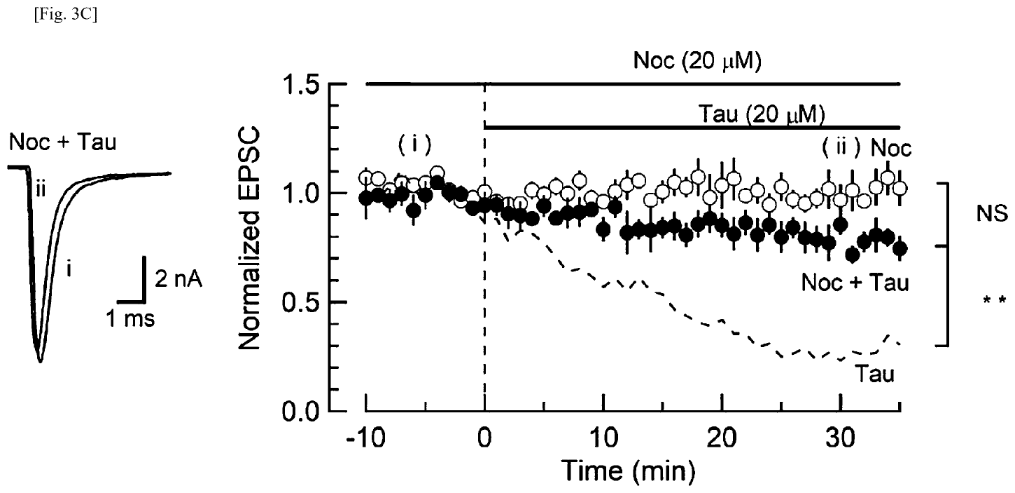

Since new MT assembly might take place after h-tau loading (FIG. 1, FIG. 1-figure supplement 1), we tested whether the tubulin polymerization blocker nocodazole might reverse the toxic effects of h-tau on SV endocytosis and synaptic transmission. In tubulin polymerization assays, nocodazole inhibited h-tau-dependent MT assembly in a concentration-dependent manner, with a maximal inhibition reached at 20 μM (FIG. 3A). In presynaptic capacitance measurements, nocodazole (20 μM) co-loaded with h-tau (20 μM) fully prevented the h-tau toxicities on endocytosis (FIG. 3B) and synaptic transmission (FIG. 3C). Nocodazole alone (20 μM) had no effect on exo-endocytosis (FIG. 3B) or EPSC amplitude (FIG. 3C). It is highly likely that WT h-tau loaded in calyceal terminals newly assembled MTs, thereby impairing SV endocytosis and synaptic transmission.

WT h-Tau Assembles MTs and Sequesters Dynamins in Calyceal Terminals

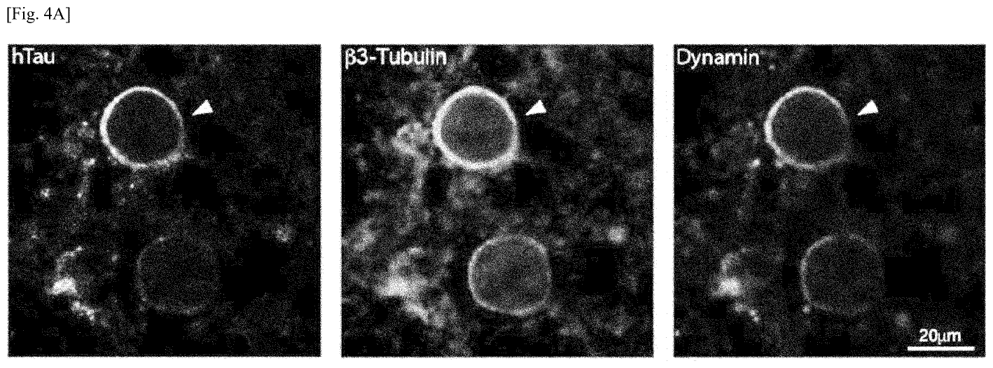

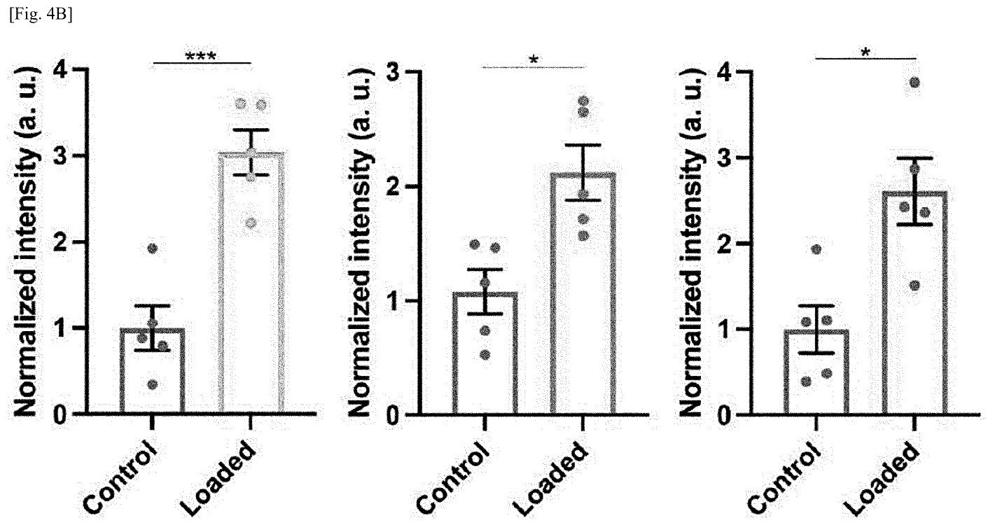

The monomeric GTPases dynamin 1 and 3 play critical roles in the endocytic fission of SVs (50-52). Since dynamin is originally discovered as a MT-binding protein (39), we hypothesized that newly assembled MTs might trap free dynamins in cytosol. If this is the case, MT-bound form of dynamin would be increased. To test this hypothesis, we performed immunofluorescence microscopy and image analysis to quantify MTs and dynamin. After whole-cell infusion of h-tau into calyceal terminals, slices were chemically fixed and permeabilized to allow cytosolic free molecules such as tubulin monomers to be washed out of the terminal, thereby enhancing the signals from large structures such as MTs or MT-bound molecules. Fluorescent h-tau antibody identified calyceal terminals loaded with WT h-tau (20 μM, FIG. 4A). Double staining with mouse β3-tubulin antibody revealed a 2.1-fold increase in MT signals in h-tau-loaded terminals, compared with those without h-tau loading (p=0.01, n=5, two-tailed unpaired t-test with Welch's correction, FIG. 4B). Triple labeling with dynamin antibodies further revealed a 2.6-fold increase in dynamin signal (p=0.01, n=5, two-tailed t-test with Welch's correction, FIG. 4B). In super-resolution imaging, dynamins are shown in clusters along MTs in tau-loaded calyceal terminal (FIG. 4-figure supplement 1). These results suggest that soluble WT h-tau can assemble MTs in presynaptic terminals, thereby sequestering cytosolic dynamins that are indispensable for SV endocytosis.

Besides dynamins, MTs can bind to various other proteins. Among them, formin mDia can bind to MTs (53) and involved in the endocytic scaffold functions together with F-actin, intersectin and endophilin. Although acute depolymerization of F-actin (38, 46) or genetic ablation of intersectin (54) has no effect on SV endocytosis at the calyx of Held, the formin mDia inhibitor SMFH2 reportedly inhibits endocytosis at the calyx terminals in pre-hearing rats (postnatal day [P]8-12) (55). We re-examined whether the drug might inhibit SV endocytosis at calyceal terminals in slices from post-hearing mice (P13-14). SMFH2 slightly prolonged SV endocytosis, but this effect was statistically insignificant (FIG. 4-figure supplement 2A). Thus, formin unlikely makes substantial contribution to the marked endocytic slowing observed after intra-terminal tau loading (FIG. 2).

It may also be argued that binding of endophilin to MTs (56) might cause EPSC rundown since endophilin is involved in clathrin uncoating (57), which is required for SV refilling with glutamate. If SV refilling during recycling is impaired, miniature EPSCs are decreased in amplitude and frequency (58). However, neither amplitude nor frequency was affected by intra-terminal loading of tau (20 μM) (FIG. 4-figure supplement 2B). Thus, endophilin-MT binding unlikely underlies EPSC rundown by intra-terminal tau infusion (FIG. 1).

A microtubule-dynamin binding inhibitor peptide attenuates h-tau toxicities on SV endocytosis and synaptic transmission

To prevent toxic effects of h-tau on endocytosis and transmission, we searched for a dominant-negative (DN) peptide blocking MT-dynamin binding. Since the MT binding domain of dynamins is unknown, we synthesized 11 peptides from the pleckstrin-homology (PH) domain and 11 peptides from the proline-rich domain of dynamin 1 (FIG. 4—figure supplement 1A) and submitted them to the MT-dynamin1 binding assay. Out of 22 peptides, one peptide corresponding to the amino acid sequence 560-571 of PH domain, which we named “PHDP5”, significantly inhibited the MT-dynamin 1 interaction (FIG. 5A, Figures5-figure supplement 1B and C). By SYPRO orange staining, dynamin 1 is found as a ˜100 kDa band, 1.7±0.4% in precipitates (ppts). In the presence of MT, dynamin 1 in ppts increased to 22.6±2.4%, indicating sequestration of dynamin 1 by MTs. When PHDP5 was added to MT and dynamin 1, dynamin1 in the ppt fraction decreased to 6.3±2.4%, indicating that PHDP5 works as a DN peptide for inhibiting MT-dynamin interactions (FIG. 5A).

A cryo-electron microscope study on dynamin 1 assembled on lipid membrane has revealed that PH domain is tucked up into dynamin structure in apo state, but upon GTP binding, exposed toward membrane by a conformational change (59). In negatively stained electron micrographs, dynamin 1 is periodically arranged on the surface of MTs (60), suggesting a helical polymerization like in dynamin-membrane interaction (61). Therefore, PH domain including the putative binding site PHDP5 is likely exposed toward MT surface. To examine whether PH domain of dynamin 1 can directly bind to MTs, immunofluorescence labelled MTs and glutathione transferase-tagged PH domain (GST-PH) were mixed and observed by confocal and electron microscopy (FIG. 5-figure supplement 2). In confocal microscopic imaging, GST-PH co-localized with MTs, in contrast to controls, where MTs were mixed with GST alone (FIG. 5-figure supplement 2A). These results were further confirmed in electron microscopic imaging, showing co-localizations of MTs and GST-PH (FIG. 5-figure supplement 2B). Thus, dynamin 1 PH domain can associate with MTs, although it remains to be determined whether PHDP5 can directly bind to MTs.

Loading of PHDP5 (0.25 mM) alone in calyceal terminals had no effect on exo-endocytic capacitance changes, but when co-loaded with WT h-tau (20 μM), it significantly attenuated the h-tau-induced endocytic slowing (p<0.05, FIG. 5B). Scrambled PHDP5 peptide (0.25 mM) loaded as a control had no effect on h-tau-induced endocytic slowing. Like its effect on capacitance changes, intra-terminal infusion of PHDP5 alone (1 mM) did not affect EPSC amplitude, but when co-loaded with WT h-tau (20 μM), significantly attenuated the inhibitory effect of h-tau on EPSC amplitude (p<0.01, FIG. 5C). Co-infusion of scrambled PHDP5 (1 mM) with h-tau (20 μM) did not affect the h-tau-induced EPSC rundown (p=0.46). These results further support that WT h-tau causes dynamin deficiency via new assembly of MTs thereby impairing SV endocytosis and synaptic transmission. These results also highlight PHDP5 as a potential therapeutic tool for rescuing synaptic dysfunctions associated with AD or PD.

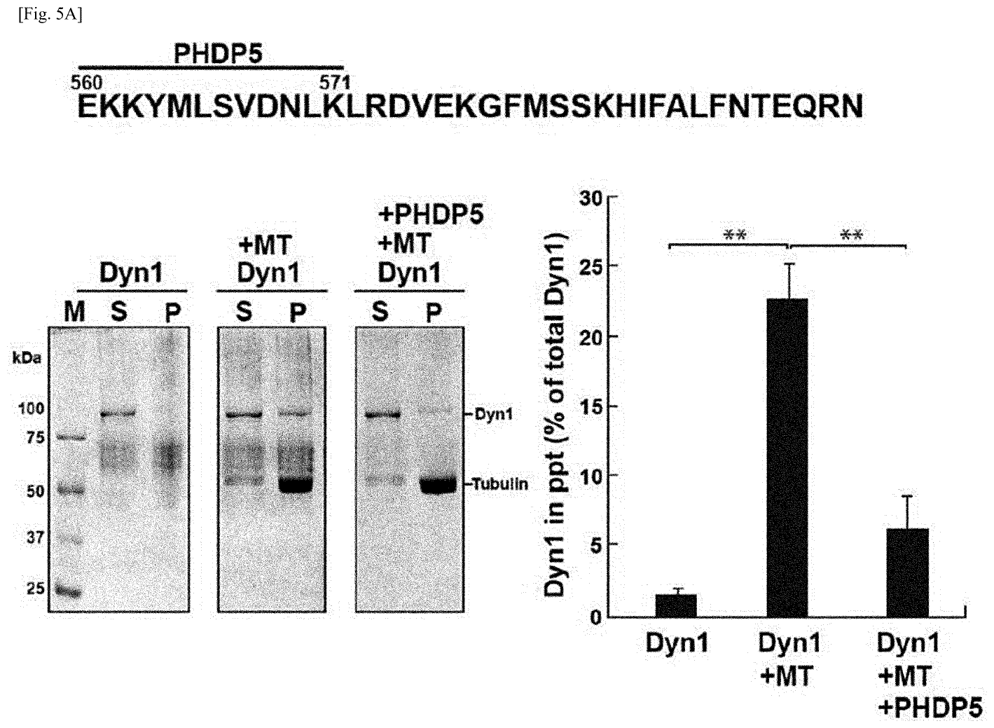

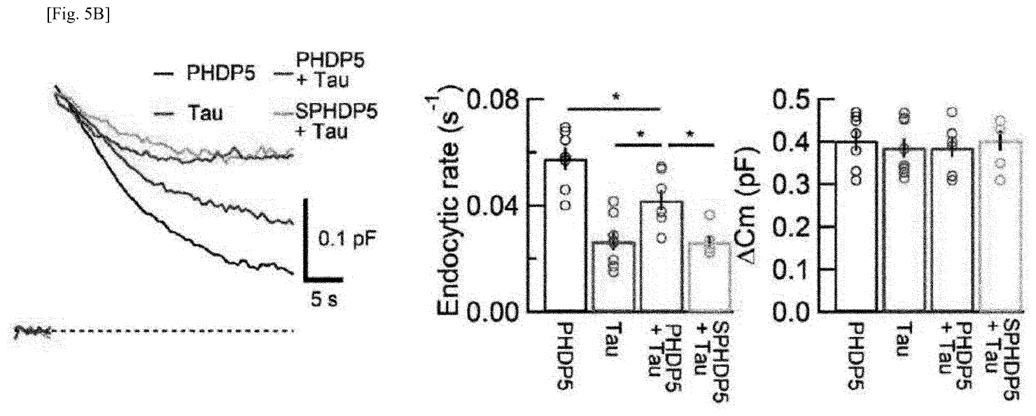

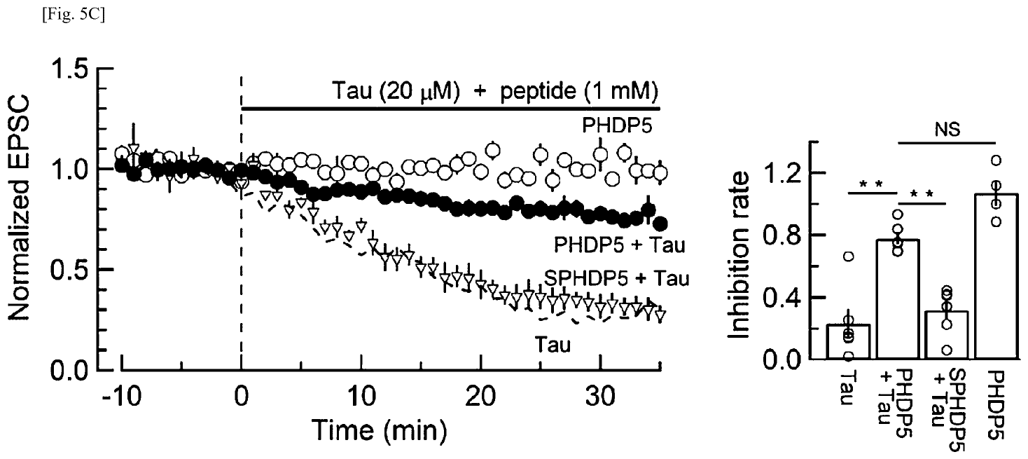

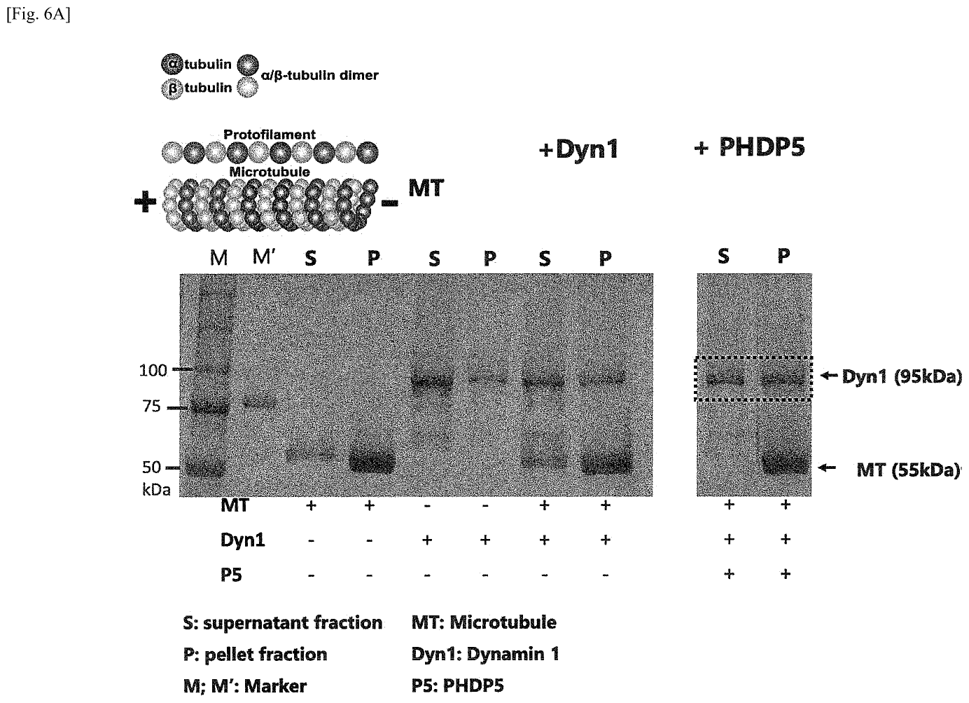

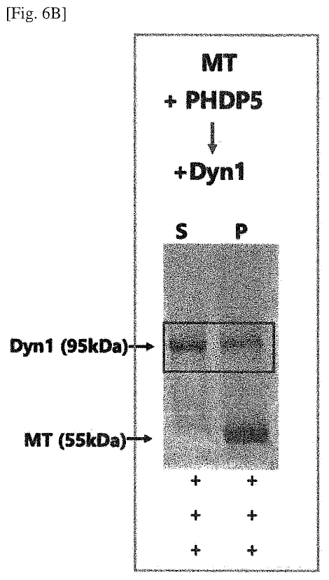

MTs were prepared from tubulin and stabilized by taxol. And then taxol stabilized MTs and Dyn1 were incubated with/without PHDP5. Using ultracentrifugation to separate supernatant (unbinding protein) and pellet (bound protein) fractions. Samples were analyzed by SDS-PAGE gel and visualized by SYPRO Orange staining. Results showed % of Dyn1 in pellet fraction has no difference between supernatant and pellet fraction after incubation with PHDP5. This may cause by competitive binding of Dyn1/PHDP5 to MTs (FIG. 6A).

We changed the incubation order that MTs were mixed with PHDP5 and then incubated with Dyn1. Results showed % of Dyn1 was decreased in pellet fraction under PHDP5 existing (FIG. 6B).

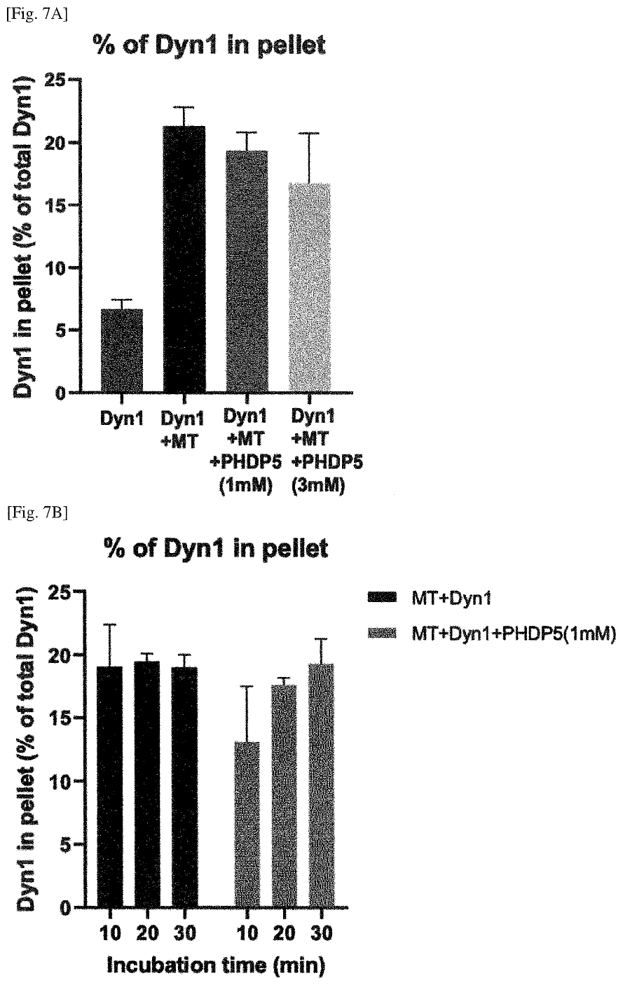

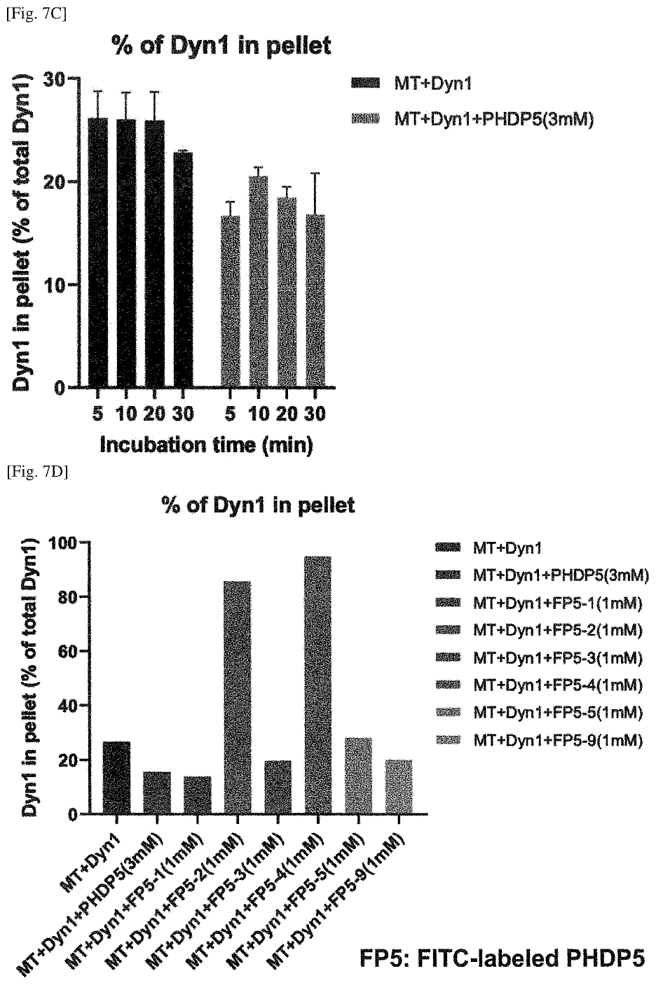

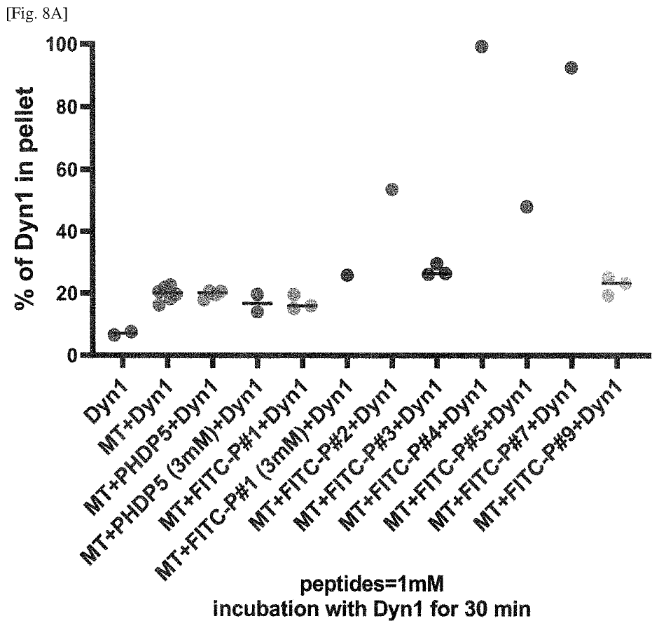

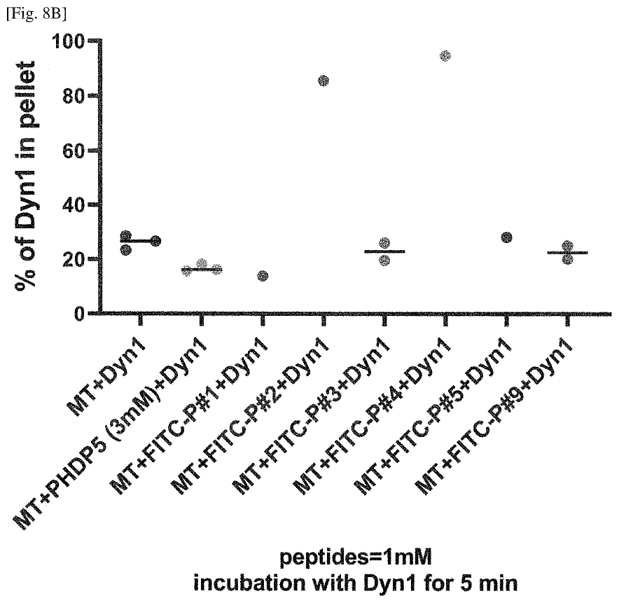

For optimizing the condition of PHDP5 to block MT-dynamin binding, we have tried different PHDP5 concentration and incubation period with Dyn1. The results showed 9% of Dyn1 in pellet fraction was lower when MTs incubated with 3 mM of PHDP5 than 1 mM. Lower % of Dyn1 in pellet fraction reveals the potential ability to block MT-dynamin interaction (FIG. 7A). When MTs incubated with 1 mM of PHDP5, shorter incubation period with Dyn1 (10 min) showed lower % of Dyn1 in pellet fraction. Without PHDP5, there is no difference between each time point (FIG. 7B). When MTs incubated with 3 mM of PHDP5, incubation with Dyn1 for 5 min showed lower % of Dyn1 in pellet fraction (FIG. 7C). We prepared several FITC-labeled PHDP5 peptide candidates to improve the brain barrier permeability of PHDP5 (Table 3). The N-terminus of the construct was protected with β-alanine (BA) and labeled with FITC. Under the same condition, several FITC-labeled PHDP5 candidates and its' scrambled control were also tested. No. 1 candidate (SQID No. 16) showed lower % of Dyn1 than PHDP5 (FIG. 7D). The results of statistical analysis were shown in FIGS. 8A and 8B.

TABLE 3

| No. | | Peptide Sequence | Mol. Mass | Mol. Formula | SQID No. |
|---|---|---|---|---|---|
| 1 | FP5#1 | (FITC)βA A E K K Y M L S V D N L | 3537.84 | C156H248N52O39S2 | 16 |
| | | K Y G R K K R R Q R R R-amide | | | |
| 2 | FP5#2 | (FITC)βA A E K K Y M L S V D N L | 3493.87 | C166H260N36O42S2 | 17 |
| | | K A A V A L L P A V L L A L L A | | | |
| | | P-amide | | | |
| 3 | FP5#3 | (FITC)βA A E K K Y M L S V D N L | 3079.47 | C138H210N34O42S2 | 18 |
| | | K A A A A E K A A A A E K-amide | | | |
| 4 | FP5#4 | (FITC)βA A E K K Y M L S V D N L | 4224.18 | C196H298N54O45S3 | 19 |
| | | K R Q I K I W F Q N R R M K W K | | | |
| | | K-amide | | | |
| 5 | FP5#5 | (FITC)βA A E K K Y M L S V D N L | 3125.63 | C148H228N32O38S2 | 20 |
| | | K A A V L L P V L L A A P-amide | | | |
| 6 | FP5#6 | (FITC)βA A E K K Y M L S V D N L | 3374.78 | C152H231N45O39S2 | 21 |
| | | K R R L S Y S R R R F-amide | | | |
| 7 | FP5#7 | (FITC)βA A K V L N D E S K M Y L | 3537.84 | C156H248N52O39S2 | 22 |
| | | K Y G R K K R R Q R R R-amide | | | |
| 8 | FP5#8 | (FITC)βA A K V L N D E S K M Y L | 3493.87 | C166H260N36O42S2 | 23 |
| | | K A A V A L L P A V L L A L L A | | | |
| | | P-amide | | | |
| 9 | FP5#9 | (FITC)βA A K V L N D E S K M Y L | 3079.47 | C138H210N34O42S2 | 24 |
| | | K A A A A E K A A A A E K-amide | | | |
| 10 | FP5#10 | (FITC)βA A K V L N D E S K M Y L | 4224.18 | C196H298N54O45S3 | 25 |
| | | K R Q I K I W F Q N R R M K W K | | | |
| | | K-amide | | | |
| 11 | FP5#11 | (FITC)βA A K V L N D E S K M Y L | 3125.63 | C148H228N32O38S2 | 26 |
| | | K A A V L L P V L L A A P-amide | | | |
| 12 | FP5#12 | (FITC)βA A K V L N D E S K M Y L | 3374.78 | C152H231N45O39S2 | 27 |
| | | K R R L S Y S R R R F-amide | | | |

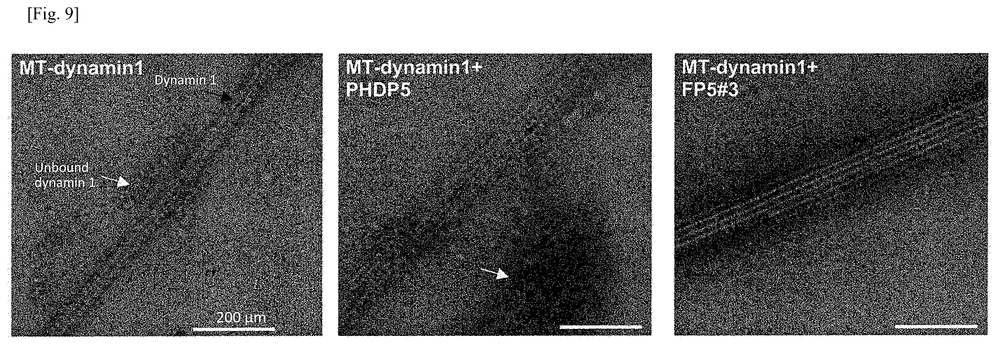

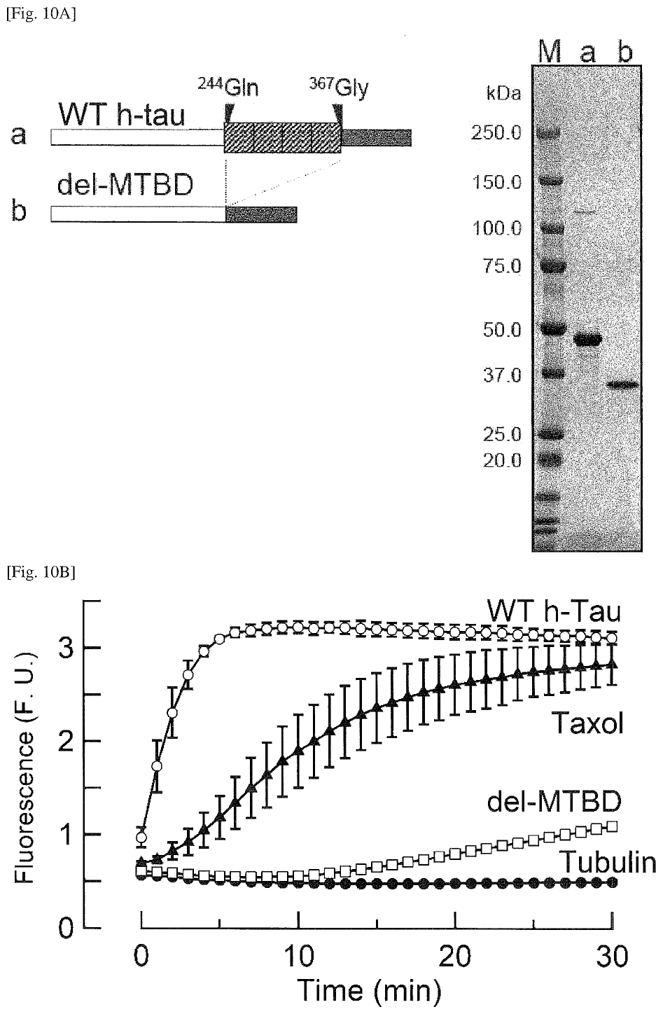

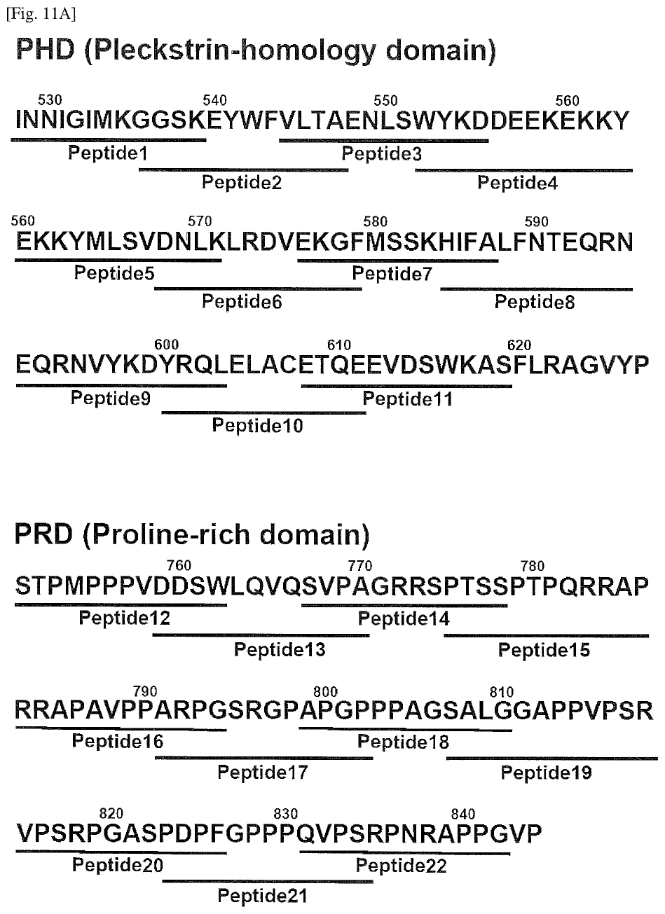

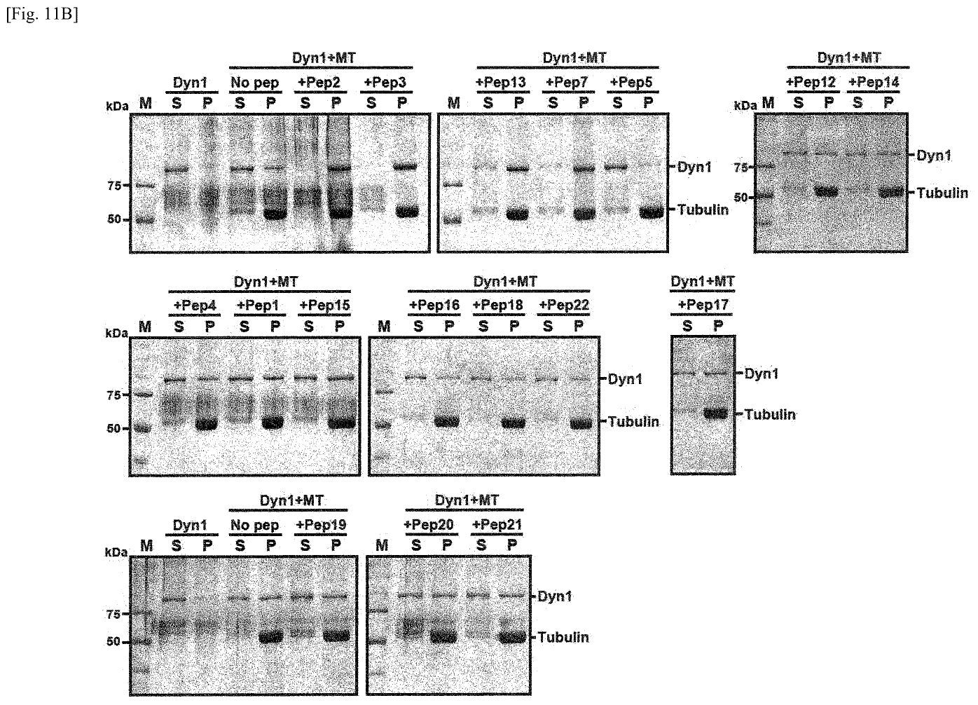

Electron images of negatively stained microtubules with dynamin1 in the presence or absence of PHDP5 are shown in FIG. 9. MT-dynamin1+FP5 #3 seems to stabilize MT-dynamin 1 interaction.

### DISCUSSION

Using the calyx of Held in brainstem slices as an AD model for dissecting mammalian central excitatory synaptic transmission, we demonstrated that intra-terminal loading of WT h-tau impairs vesicle endocytosis and synaptic transmission via de novo MT assembly. Previous over-expression studies in cultured cells reported MT assembly by injection or overexpression of WT tau (36, 62, 63) or phosphorylated tau (34, 64). Compared with overexpression, our whole-cell method allows targeted loading of molecules in presynaptic terminals at defined concentrations because of a large pipette-to-cell volume ratio (44). In postmortem brain tissue homogenates from AD patients, soluble tau content is estimated as 6 ng/μg of protein, which is 8 times higher than controls (65). Assuming protein contents in brain homogenate as 10%, 60 kDa tau concentration in AD patients' brain is estimated as 10 μM. Since elevation of soluble tau concentration likely occurs mainly in axons and axon terminal compartments of neurons, soluble tau concentration in AD patients in presynaptic terminals can be higher than that. Our results at the calyx of Held suggest that excitatory synaptic transmission, in general, can be significantly impaired in such situations. In fact, the magnitude of EPSC rundown after WT h-tau loading is comparable to that caused by the clinical dose of general anesthetic isoflurane at the calyx of Held in slice (48). In AD, tau pathology starts from the locus coeruleus in the brainstem and undergoes trans-synaptic propagation to hippocampal and neocortical neurons (66). Present results in our model synapse suggest that synaptic functions in such tau-propagation pathways can be severely affected at the early stage of AD.

Membrane capacitance measurements at the calyx of Held revealed the primary target of WT h-tau toxicity as SV endocytosis. Endocytic slowing impairs SV recycling and reuse, thereby inhibiting SV exocytosis, particularly in response to high-frequency stimulations (49). The toxic effects of h-tau on SV endocytosis and synaptic transmission were prevented by nocodazole co-application. Together with the lack of toxicity of del-MTBD and toxic effects of taxol on synaptic transmission, these results suggest pathological roles of over-assembled MTs. Like WT h-tau, intra-terminal loading of WT α-synuclein slows SV endocytosis and impairs fidelity of high-frequency neurotransmission at the calyx of Held (46). α-Synuclein toxicities can be rescued by blocking MT assembly with nocodazole or a photosensitive colchicine derivative PST-1. Thus, a common mechanism likely underlies synaptic dysfunctions in AD and PD. Compared with α-synuclein, h-tau toxicity is much stronger on endocytosis as well as on synaptic transmission. Thus, abnormal elevation of endogenous molecules beyond homeostatic level may cause AD and PD symptoms, like many other human diseases.

Although the GTPase dynamin is a well-known player in endocytic fission of SVs (50, 52), it was originally discovered as a MT-binding protein (39). Subsequent studies indicated that this interaction upregulates dynamin's GTPase activity (67, 68) and can induce MT instability with dynamin 2 (69) or stabilizes MT bundle formation with dynamin 1 (60). However, the binding domain of dynamin remained unidentified. In this study, calyceal terminals loaded with WT h-tau showed a prominent increase in immunofluorescence signal intensity corresponding to bound dynamins. This was associated with an elevation in intra-terminal MTs, suggesting that newly assembled MTs induced by loaded h-tau sequestered cytosolic dynamins. These results well explain impairments of SV endocytosis by intra-terminal h-tau loading. Through synthetic peptide screening, we found that a dodecapeptide from dynamin 1 PH domain significantly inhibited the MT-dynamin interaction. This peptide PHDP5 is ˜80% homologous to dynamin 3, another isoform involved in vesicle endocytosis (51). Although direct binding of this peptide to MTs remains to be seen, it significantly rescued endocytic impairments and EPSC rundown induced by intra-terminal WT h-tau. Hence, MTs over-assembled by soluble WT h-tau proteins likely sequester free dynamins in presynaptic terminals, thereby blocking SV endocytosis and synaptic transmission, at least in this slice model. This dynamin sequestration mechanism by newly assembled MTs may also underlie the toxic effect of α-synuclein on SV endocytosis (46) in PD.

Unlike WT h-tau, the FTDP-linked mutant tau does not affect SV endocytosis (32), but binds to both actin filaments (70) and the SV transmembrane protein synaptogyrin (31), thereby immobilizing SVs (31, 32). WT-tau can also bind to synaptogyrin (31), but cannot bind to F-actins because of a difference in the MT-binding regions between FTDP mutant and WT tau (62, 71). However, WT h-tau can bind to various other macromolecules and organelles such as MTs, neurofilaments and ribosomes (72) as well as to synaptogyrin, thereby possibly immobilizing SVs. Recycling transport of SVs impaired by this mechanism might additionally contribute to the rundown of synaptic transmission remaining unblocked by the MT-dynamin blocker peptide. In the absence of a powerful tool for alleviating symptoms associated with AD or PD, the calyx of Held slice model might provide a platform upon which therapeutic tools for rescuing synaptic dysfunctions can be pursued. The combination of this slice model with animal models could provide a new pathway toward rescuing neurological disorders.

### REFERENCES

1. K. R. Brunden, J. Q. Trojanowski, V. M. Lee, Advances in tau-focused drug discovery for Alzheimer's disease and related tauopathies. Nat Rev Drug Discov 8, 783-793 (2009).
2. V. M. Lee, K. R. Brunden, M. Hutton, J. Q. Trojanowski, Developing therapeutic approaches to tau, selected kinases, and related neuronal protein targets. Cold Spring Harb Perspect Med 1, a006437 (2011).
3. L. I. Binder, A. Frankfurter, L. I. Rebhun, The distribution of tau in the mammalian central nervous system. J Cell Biol 101, 1371-1378 (1985).
4. A. Kubo et al., Distribution of endogenous normal tau in the mouse brain. J Comp Neurol 527, 985-998 (2019).
5. J. Biernat, N. Gustke, G. Drewes, E. M. Mandelkow, E. Mandelkow, Phosphorylation of Ser262 strongly reduces binding of tau to microtubules: distinction between PHF-like immunoreactivity and microtubule binding. Neuron 11, 153-163 (1993).
6. D. N. Drechsel, A. A. Hyman, M. H. Cobb, M. W. Kirschner, Modulation of the dynamic instability of tubulin assembly by the microtubule-associated protein tau. Mol Biol Cell 3, 1141-1154 (1992).
7. C. Ballatore, V. M. Lee, J. Q. Trojanowski, Tau-mediated neurodegeneration in Alzheimer's disease and related disorders. Nat Rev Neurosci 8, 663-672 (2007).
8. M. G. Spillantini, M. Goedert, Tau pathology and neurodegeneration. Lancet Neurol 12, 609-622 (2013).
9. S. G. Greenberg, P. Davies, A preparation of Alzheimer paired helical filaments that displays distinct tau proteins by polyacrylamide gel electrophoresis. Proc Natl Acad Sci US A 87, 5827-5831 (1990).
10. I. Grundke-Iqbal et al., Abnormal phosphorylation of the microtubule-associated protein tau (tau) in Alzheimer cytoskeletal pathology. Proc Natl Acad Sci USA 83, 4913-4917 (1986).
11. H. Yoshida, Y. Ihara, Tau in paired helical filaments is functionally distinct from fetal tau: assembly incompetence of paired helical filament-tau. J Neurochem 61, 1183-1186 (1993).
12. M. Morris, S. Maeda, K. Vossel, L. Mucke, The many faces of tau. Neuron 70, 410-426 (2011).
13. J. Gotz, L. M. Ittner, M. Fandrich, N. Schonrock, Is tau aggregation toxic or protective: a sensible question in the absence of sensitive methods? J Alzheimers Dis 14, 423-429 (2008).
14. D. J. Koss et al., Soluble pre-fibrillar tau and beta-amyloid species emerge in early human Alzheimer's disease and track disease progression and cognitive decline. Acta Neuropathol 132, 875-895 (2016).
15. A. Harada et al., Altered microtubule organization in small-calibre axons of mice lacking tau protein. Nature 369, 488-491 (1994).
16. K. A. Vossel et al., Tau reduction prevents Abeta-induced defects in axonal transport. Science 330, 198 (2010).
17. A. Yuan, A. Kumar, C. Peterhoff, K. Duff, R. A. Nixon, Axonal transport rates in vivo are unaffected by tau deletion or overexpression in mice. J Neurosci 28, 1682-1687 (2008).
18. L. M. Ittner et al., Dendritic function of tau mediates amyloid-beta toxicity in Alzheimer's disease mouse models. Cell 142, 387-397 (2010).
19. E. D. Roberson et al., Reducing endogenous tau ameliorates amyloid beta-induced deficits in an Alzheimer's disease mouse model. Science 316, 750-754 (2007).
20. J. A. Fein et al., Co-localization of amyloid beta and tau pathology in Alzheimer's disease synaptosomes. Am J Pathol 172, 1683-1692 (2008).
21. H. C. Tai et al., The synaptic accumulation of hyperphosphorylated tau oligomers in Alzheimer disease is associated with dysfunction of the ubiquitin-proteasome system. Am J Pathol 181, 1426-1435 (2012).
22. A. de Calignon et al., Propagation of tau pathology in a model of early Alzheimer's disease. Neuron 73, 685-697 (2012).
23. L. Liu et al., Trans-synaptic spread of tau pathology in vivo. PLOS One 7, e31302 (2012).
24. A. M. Pooler, E. C. Phillips, D. H. Lau, W. Noble, D. P. Hanger, Physiological release of endogenous tau is stimulated by neuronal activity. EMBO Rep 14, 389-394 (2013).
25. K. Yamada et al., Neuronal activity regulates extracellular tau in vivo. J Exp Med 211, 387-393 (2014).
26. J. L. Guo, V. M. Lee, Seeding of normal Tau by pathological Tau conformers drives pathogenesis of Alzheimer-like tangles. J Biol Chem 286, 15317-15331 (2011).
27. M. Polydoro et al., Soluble pathological tau in the entorhinal cortex leads to presynaptic deficits in an early Alzheimer's disease model. Acta Neuropathol 127, 257-270 (2014).
28. A. Sydow et al., Tau-induced defects in synaptic plasticity, learning, and memory are reversible in transgenic mice after switching off the toxic Tau mutant. J Neurosci 31, 2511-2525 (2011).
29. Y. Yoshiyama et al., Synapse loss and microglial activation precede tangles in a P301S tauopathy mouse model. Neuron 53, 337-351 (2007).
30. K. Santacruz et al., Tau suppression in a neurodegenerative mouse model improves memory function. Science 309, 476-481 (2005).
31. J. McInnes et al., Synaptogyrin-3 Mediates Presynaptic Dysfunction Induced by Tau. Neuron 97, 823-835 e828 (2018).
32. L. Zhou et al., Tau association with synaptic vesicles causes presynaptic dysfunction. Nat Commun 8, 15295 (2017).
33. A. Ebneth et al., Overexpression of tau protein inhibits kinesin-dependent trafficking of vesicles, mitochondria, and endoplasmic reticulum: implications for Alzheimer's disease. J Cell Biol 143, 777-794 (1998).
34. K. Shahpasand et al., Regulation of mitochondrial transport and inter-microtubule spacing by tau phosphorylation at the sites hyperphosphorylated in Alzheimer's disease. J Neurosci 32, 2430-2441 (2012).
35. K. Stamer, R. Vogel, E. Thies, E. Mandelkow, E. M. Mandelkow, Tau blocks traffic of organelles, neurofilaments, and APP vesicles in neurons and enhances oxidative stress. J Cell Biol 156, 1051-1063 (2002).
36. E. Thies, E. M. Mandelkow, Missorting of tau in neurons causes degeneration of synapses that can be rescued by the kinase MARK2/Par-1. J Neurosci 27, 2896-2907 (2007).
37. A. Mudher et al., GSK-3beta inhibition reverses axonal transport defects and behavioural phenotypes in Drosophila. Mol Psychiatry 9, 522-530 (2004).
38. L. Piriya Ananda Babu, H. Y. Wang, K. Eguchi, L. Guillaud, T. Takahashi, Microtubule and Actin Differentially Regulate Synaptic Vesicle Cycling to Maintain High-Frequency Neurotransmission. J Neurosci 40, 131-142 (2020).
39. H. S. Shpetner, R. B. Vallee, Identification of dynamin, a novel mechanochemical enzyme that mediates interactions between microtubules. Cell 59, 421-432 (1989).
40. C. Xie et al., The homologous carboxyl-terminal domains of microtubule-associated protein 2 and TAU induce neuronal dysfunction and have differential fates in the evolution of neurofibrillary tangles. PLOS One 9, c89796 (2014).
41. S. Maeda et al., Granular tau oligomers as intermediates of tau filaments. Biochemistry 46, 3856-3861 (2007).
42. T. Hori, Y. Takai, T. Takahashi, Presynaptic mechanism for phorbol ester-induced synaptic potentiation. J Neurosci 19, 7262-7267 (1999).
43. T. Takahashi, T. Hori, Y. Nakamura, T. Yamashita, Patch-Clamp Recording Method in Slices for Studying Presynaptic Mechanisms. 137-145 (2012). 44. M. Pusch, E. Neher, Rates of diffusional exchange between small cells and a measuring patch pipette. Pflugers Arch 411, 204-211 (1988).
45. S. Kar, J. Fan, M. J. Smith, M. Goedert, L. A. Amos, Repeat motifs of tau bind to the insides of microtubules in the absence of taxol. EMBO J 22, 70-77 (2003).
46. K. Eguchi et al., Wild-Type Monomeric alpha-Synuclein Can Impair Vesicle Endocytosis and Synaptic Fidelity via Tubulin Polymerization at the Calyx of Held. J Neurosci 37, 6043-6052 (2017).
47. J. Y. Sun, L. G. Wu, Fast kinetics of exocytosis revealed by simultaneous measurements of presynaptic capacitance and postsynaptic currents at a central synapse. Neuron 30, 171-182 (2001).
48. H. Y. Wang, K. Eguchi, T. Yamashita, T. Takahashi, Frequency-Dependent Block of Excitatory Neurotransmission by Isoflurane via Dual Presynaptic Mechanisms. J Neurosci 40, 4103-4115 (2020).
49. T. Yamashita, T. Hige, T. Takahashi, Vesicle endocytosis requires dynamin-dependent GTP hydrolysis at a fast CNS synapse. Science 307, 124-127 (2005).
50. J. E. Hinshaw, S. L. Schmid, Dynamin self-assembles into rings suggesting a mechanism for coated vesicle budding. Nature 374, 190-192 (1995).
51. A. Raimondi et al., Overlapping role of dynamin isoforms in synaptic vesicle endocytosis. Neuron 70, 1100-1114 (2011).
52. K. Takei, P. S. McPherson, S. L. Schmid, P. De Camilli, Tubular membrane in-vaginations coated by dynamin rings are induced by GTP-gamma S in nerve terminals. Nature 374, 186-190 (1995).
53. F. Bartolini, G. G. Gundersen, Formins and microtubules. Biochim Biophys Acta 1803, 164-173 (2010).
54. T. Sakaba et al., Fast neurotransmitter release regulated by the endocytic scaffold intersectin. Proc Natl Acad Sci USA 110, 8266-8271 (2013).
55. T. Soykan et al., Synaptic Vesicle Endocytosis Occurs on Multiple Timescales and Is Mediated by Formin-Dependent Actin Assembly. Neuron 93, 854-866 e854 (2017). 56. K. R. Schuske et al., Endophilin is required for synaptic vesicle endocytosis by localizing synaptojanin. Neuron 40, 749-762 (2003).
57. S. Watanabe et al., Synaptojanin and Endophilin Mediate Neck Formation during Ultrafast Endocytosis. Neuron 98, 1184-1197 e1186 (2018).
58. C. Takami, K. Eguchi, T. Hori, T. Takahashi, Impact of vesicular glutamate leakage on synaptic transmission at the calyx of Held. J Physiol 595, 1263-1271 (2017).
59. L. Kong et al., Cryo-EM of the dynamin polymer assembled on lipid membrane. Nature 560, 258-262 (2018).
60. T. M. La et al., Dynamin 1 is important for microtubule organization and stabilization in glomerular podocytes. FASEB J 34, 16449-16463 (2020).
61. P. Zhang, J. E. Hinshaw, Three-dimensional reconstruction of dynamin in the constricted state. Nat Cell Biol 3, 922-926 (2001).
62. D. G. Drubin, M. W. Kirschner, Tau protein function in living cells. J Cell Biol 103, 2739-2746 (1986).
63. O. A. Shemesh, H. Erez, I. Ginzburg, M. E. Spira, Tau-induced traffic jams reflect organelles accumulation at points of microtubule polar mismatching. Traffic 9, 458-471 (2008).
64. F. Liu et al., Site-specific effects of tau phosphorylation on its microtubule assembly activity and self-aggregation. Eur J Neurosci 26, 3429-3436 (2007).
65. S. Khatoon, I. Grundke-Iqbal, K. Iqbal, Brain levels of microtubule-associated protein tau are elevated in Alzheimer's disease: a radioimmuno-slot-blot assay for nanograms of the protein. J Neurochem 59, 750-753 (1992).
66. M. Goedert, B. Falcon, F. Clavaguera, M. Tolnay, Prion-like mechanisms in the pathogenesis of tauopathies and synucleinopathies. Curr Neurol Neurosci Rep 14, 495 (2014).
67. K. Maeda, T. Nakata, Y. Noda, R. Sato-Yoshitake, N. Hirokawa, Interaction of dynamin with microtubules: its structure and GTPase activity investigated by using highly purified dynamin. Mol Biol Cell 3, 1181-1194 (1992).
68. H. S. Shpetner, R. B. Vallee, Dynamin is a GTPase stimulated to high levels of activity by microtubules. Nature 355, 733-735 (1992).
69. K. Tanabe, K. Takei, Dynamic instability of microtubules requires dynamin 2 and is impaired in a Charcot-Marie-Tooth mutant. J Cell Biol 185, 939-948 (2009).
70. T. A. Fulga et al., Abnormal bundling and accumulation of F-actin mediates tau-induced neuronal degeneration in vivo. Nat Cell Biol 9, 139-148 (2007).
71. B. Roger, J. Al-Bassam, L. Dehmelt, R. A. Milligan, S. Halpain, MAP2c, but not tau, binds and bundles F-actin via its microtubule binding domain. Curr Biol 14, 363-371 (2004).
72. T. Guo, W. Noble, D. P. Hanger, Roles of tau protein in health and disease. Acta Neuropathol 133, 665-704 (2017).
73. M. Hasegawa, M. J. Smith, M. Goedert, Tau proteins with FTDP-17 mutations have a reduced ability to promote microtubule assembly. FEBS Lett 437, 207-210 (1998).
74. H. Yamada et al., Stabilization of actin bundles by a dynamin 1/cortactin ring complex is necessary for growth cone filopodia. J Neurosci 33, 4514-4526 (2013).
75. C. Gu et al., Direct dynamin-actin interactions regulate the actin cytoskeleton. EMBO J 29, 3593-3606 (2010).
76. V. I. Slepnev, G. C. Ochoa, M. H. Butler, P. De Camilli, Tandem arrangement of the clathrin and AP-2 binding domains in amphiphysin 1 and disruption of clathrin coat function by amphiphysin fragments comprising these sites. J Biol Chem 275, 17583-17589 (2000).
77. T. Hori, T. Takahashi, Kinetics of synaptic vesicle refilling with neurotransmitter glutamate. Neuron 76, 511-517 (2012).

1-13. (canceled)

14. A method for screening for substances effective for the treatment of Alzheimer's disease, comprising a step of measuring an activity of the test substances to inhibit the binding of microtubules and dynamin 1.

15. A method for treating Alzheimer's disease comprising administering to a patient a peptide corresponding to dynamin 1.

16. The method for treating Alzheimer's disease according to claim 15, wherein the peptide corresponds to dynamin 1-pleckstrin-homology domain or dynamin 1-proline rich domain.

17. The method for treating Alzheimer's disease according to claim 15, wherein the peptide comprises an amino acid sequence selected from the group consisting of Sequence ID Numbers 7 to 9, an amino acid sequence having one or more conservative amino acid substitutions in the amino acid sequence selected from the group consisting of Sequence ID Numbers 7 to 9 or an amino acid sequence having at least 80% amino acid sequence identity with the amino acid sequence selected from the group consisting of Sequence ID Numbers 7 to 9.

18. The method for treating Alzheimer's disease according to claim 15, wherein the peptide is encapsulated in nano-particles for improved delivery of the peptide into the brain.

19. The method for treating Alzheimer's disease according to claim 15, wherein the peptide is linked to a peptide sequence that improves delivery of the peptide into the brain.

20. The method for treating Alzheimer's disease according to claim 15, wherein the peptide sequence that improves delivery of the peptide into the brain is selected from the group consisting of Sequence ID Numbers 10 to 15.

21. The method for treating Alzheimer's disease according to claim 15, wherein the peptide is fused to or conjugated with a compound that improves delivery of the peptide into the brain.

22. A method for treating Alzheimer's disease comprising administering to a patient an inhibitor of microtubule-dynamin 1 binding.


SEQUENCE LISTING (27 sequences)

SEQ ID NO: 1 - protein, 864 aa, Homo sapiens
MGNRGMEDLIPLVNRLQDAFSAIGQNADLDLPQIAVVGGQSAGKSSVLENFVGRDFLPRGSGIVTRRPLVLQLVNATTEYAEFLHCKGKKFTDFEEVRLEIEAETDRVTGTNKGISPVPINLRVYSPHVLNLTLVDLPGMTKVPVGDQPPDIEFQIRDMLMQFVTKENCLILAVSPANSDLANSDALKVAKEVDPQGQRTIGVITKLDLMDEGTDARDVLENKLLPLRRGYIGVVNRSQKDIDGKKDITAALAAERKFFLSHPSYRHLADRMGTPYLQKVLNQQLTNHIRDTLPGLRNKLQSQLLSIEKEVEEYKNFRPDDPARKTKALLQMVQQFAVDFEKRIEGSGDQIDTYELSGGARINRIFHERFPFELVKMEFDEKELRREISYAIKNIHGIRTGLFTPDMAFETIVKKQVKKIREPCLKCVDMVISELISTVRQCTKKLQQYPRLREEMERIVTTHIREREGRTKEQVMLLIDIELAYMNTNHEDFIGFANAQQRSNQMNKKKTSGNQDEILVIRKGWLTINNIGIMKGGSKEYWFVLTAENLSWYKDDEEKEKKYMLSVDNLKLRDVEKGFMSSKHIFALFNTEQRNVYKDYRQLELACETQEEVDSWKASFLRAGVYPERVGDKEKASETEENGSDSFMHSMDPQLERQVETIRNLVDSYMAIVNKTVRDLMPKTIMHLMINNTKEFIFSELLANLYSCGDQNTLMEESAEQAQRRDEMLRMYHALKEALSIIGDINTTTVSTPMPPPVDDSWLQVQSVPAGRRSPTSSPTPQRRAPAVPPARPGSRGPAPGPPPAGSALGGAPPVPSRPGASPDPFGPPPQVPSRPNRAPPGVPSRSGQASPSRPESPRPPFDL

SEQ ID NO: 2 - protein, 851 aa, Mus musculus
MGNRGMEDLIPLVNRLQDAFSAIGQNADLDLPQIAVVGGQSAGKSSVLENFVGRDFLPRGSGIVTRRPLVLQLVNSTTEYAEFLHCKGKKFTDFEEVRLEIEAETDRVTGTNKGISPVPINLRVYSPHVLNLTLVDLPGMTKVPVGDQPPDIEFQIRDMLMQFVTKENCLILAVSPANSDLANSDALKIAKEVDPQGQRTIGVITKLDLMDEGTDARDVLENKLLPLRRGYIGVVNRSQKDIDGKKDITAALAAERKFFLSHPSYRHLADRMGTPYLQKVLNQQLTNHIRDTLPGLRNKLQSQLLSIEKEVDEYKNFRPDDPARKTKALLQMVQQFAVDFEKRIEGSGDQIDTYELSGGARINRIFHERFPFELVKMEFDEKELRREISYAIKNIHGIRTGLFTPDLAFEATVKKQVQKLKEPSIKCVDMVVSELTSTIRKCSEKLQQYPRLREEMERIVTTHIREREGRTKEQVMLLIDIELAYMNTNHEDFIGFANAQQRSNQMNKKKTSGNQDEILVIRKGWLTINNIGIMKGGSKEYWFVLTAENLSWYKDDEEKEKKYMLSVDNLKLRDVEKGFMSSKHIFALFNTEQRNVYKDYRQLELACETQEEVDSWKASFLRAGVYPERVGDKEKASETEENGSDSFMHSMDPQLERQVETIRNLVDSYMAIVNKTVRDLMPKTIMHLMINNTKEFIFSELLANLYSCGDQNTLMEESAEQAQRRDEMLRMYHALKEALSIIGDINTTTVSTPMPPPVDDSWLQVQSVPAGRRSPTSSPTPQRRAPAVPPARPGSRGPAPGPPPAGSALGGAPPVPSRPGASPDPFGPPPQVPSRPNRAPPGVPRITISDP

SEQ ID NO: 3 - protein, 110 aa, Homo sapiens
VIRKGWLTINNIGIMKGGSKEYWFVLTAENLSWYKDDEEKEKKYMLSVDNLKLRDVEKGFMSSKHIFALFNTEQRNVYKDYRQLELACETQEEVDSWKASFLRAGVYPER

SEQ ID NO: 4 - protein, 110 aa, Mus musculus; identical to SEQ ID NO: 3

SEQ ID NO: 5 - protein, 94 aa, Homo sapiens
STPMPPPVDDSWLQVQSVPAGRRSPTSSPTPQRRAPAVPPARPGSRGPAPGPPPAGSALGGAPPVPSRPGASPDPFGPPPQVPSRPNRAPPGVP

SEQ ID NO: 6 - protein, 94 aa, Mus musculus; identical to SEQ ID NO: 5

SEQ ID NO: 7 - protein, 12 aa, synthetic construct
EKKYMLSVDNLK

SEQ ID NO: 8 - protein, 12 aa, synthetic construct
PTSSPTPQRRAP

SEQ ID NO: 9 - protein, 12 aa, synthetic construct
RRAPAVPPARPG

SEQ ID NO: 10 - protein, 11 aa, synthetic construct
YGRKKRRQRRR

SEQ ID NO: 11 - protein, 16 aa, synthetic construct
AAVALLPAVLLALLAP

SEQ ID NO: 12 - protein, 12 aa, synthetic construct
AAAAEKAAAAEK

SEQ ID NO: 13 - protein, 16 aa, synthetic construct
RQIKIWFQNRRMKWKK

SEQ ID NO: 14 - protein, 12 aa, synthetic construct
AAVLLPVLLAAP

SEQ ID NO: 15 - protein, 10 aa, synthetic construct
RRLSYSRRRF

SEQ ID NO: 16 - protein, 25 aa, synthetic construct
AAEKKYMLSVDNLKYGRKKRRQRRR

SEQ ID NO: 17 - protein, 30 aa, synthetic construct
AAEKKYMLSVDNLKAAVALLPAVLLALLAP

SEQ ID NO: 18 - protein, 26 aa, synthetic construct
AAEKKYMLSVDNLKAAAAEKAAAAEK

SEQ ID NO: 19 - protein, 30 aa, synthetic construct
AAEKKYMLSVDNLKRQIKIWFQNRRMKWKK

SEQ ID NO: 20 - protein, 26 aa, synthetic construct
AAEKKYMLSVDNLKAAVLLPVLLAAP

SEQ ID NO: 21 - protein, 24 aa, synthetic construct
AAEKKYMLSVDNLKRRLSYSRRRF

SEQ ID NO: 22 - protein, 25 aa, synthetic construct
AAKVLNDESKMYLKYGRKKRRQRRR

SEQ ID NO: 23 - protein, 30 aa, synthetic construct
AAKVLNDESKMYLKAAVALLPAVLLALLAP

SEQ ID NO: 24 - protein, 26 aa, synthetic construct
AAKVLNDESKMYLKAAAAEKAAAAEK

SEQ ID NO: 25 - protein, 30 aa, synthetic construct
AAKVLNDESKMYLKRQIKIWFQNRRMKWKK

SEQ ID NO: 26 - protein, 26 aa, synthetic construct
AAKVLNDESKMYLKAAVLLPVLLAAP

SEQ ID NO: 27 - protein, 24 aa, synthetic construct
AAKVLNDESKMYLKRRLSYSRRRF